(19) United States
(12) Patent Application Publication
(10) Pub. No.: US 2013/0144098 A1
(43) Pub. Date: Jun. 6, 2013

(54) DIRECT CONVERSION OF BIOMASS OXYGENATES TO HYDROCARBONS

(71) Applicants: Sourabh S. Pansare, Bartlesville, OK (US); Brian C. Dunn, Bartlesville, OK (US); Edgar Lotero, Cleveland, TX (US); Alexandru Platon, Bartlesville, OK (US); Leendert Arie Gerritsen, Lunteren (NL); Stephen Marshall, Bartlesville, OK (US); Kristi A. Fjare, Bartlesville, OK (US); Ronald E. Brown, Collinsville, OK (US)

(72) Inventors: Sourabh S. Pansare, Bartlesville, OK (US); Brian C. Dunn, Bartlesville, OK (US); Edgar Lotero, Cleveland, TX (US); Alexandru Platon, Bartlesville, OK (US); Leendert Arie Gerritsen, Lunteren (NL); Stephen Marshall, Bartlesville, OK (US); Kristi A. Fjare, Bartlesville, OK (US); Ronald E. Brown, Collinsville, OK (US)

(73) Assignee: PHILLIPS 66 COMPANY, Houston, TX (US)

(21) Appl. No.: 13/693,718

(22) Filed: Dec. 4, 2012

Related U.S. Application Data
(60) Provisional application No. 61/567,287, filed on Dec. 6, 2011.
(60) Provisional application No. 61/637,934, filed on Apr. 25, 2012.

(51) Int. Cl. C07C 1/24; B01D 3/00

(52) U.S. Cl. CPC C07C 1/24; B01D 3/009
USPC 585/310; 422/187; 585/733

(57) ABSTRACT

A single pass direct conversion of biomass derived oxygenates to longer chain hydrocarbons is described. The longer chain hydrocarbons include higher naphthene content which is quite useful in the distillate range fuels or more particularly, the jet and diesel range fuels. Naphthenes help the biomass derived hydrocarbons meet product specifications for jet and diesel while really helping cold flow properties.

### CROSS-REFERENCE TO RELATED APPLICATIONS

This application is a non-provisional application which claims benefit under 35 USC §119(e) to U.S. Provisional Application Ser. No. 61/567,287 filed Dec. 6, 2011, entitled “Direct Conversion of Biomass Oxygenates to Distillate-Range Hydrocarbons,” which is incorporated herein in its entirety. This application is also a non-provisional application which claims benefit under 35 USC §119(e) to U.S. Provisional Application Ser. No. 61/637,934 filed Apr. 25, 2012, also entitled “Direct Conversion of Biomass Oxygenates to Distillate-Range Hydrocarbons,” which is also incorporated herein in its entirety.

### STATEMENT REGARDING FEDERALLY SPONSORED RESEARCH

None.

### FIELD OF THE INVENTION

This invention relates to the generation of fuels from biomass.

### BACKGROUND OF THE INVENTION

Biomass represents a renewable source for the production of fungible transportation fuels and fuel oxygenates. Cellulose, hemicellulose, and lignin are the three main constituents of biomass. When the cellulose and hemicellulose portions of biomass are subjected to acid hydrolysis, the sugar polymers get converted to sugar monomers. These monomers, on subsequent hydrogenation, get converted to C6 and C5 alcohols (sorbitol and xylitol, respectively, along with other polyols and byproducts). The polyols formed can be treated through various processes to produce hydrocarbon fuels.

Several researchers have attempted to convert biomass derived oxygenates (polyols, ketones etc.) to monoalcohols and C6+ hydrocarbon fuels due to their higher fuel value. Dumesic and coworkers (US 2009/0124839; Chheda, et al., 2007; Bond, et al., 2010; Gürbüz, et al., 2010) have attempted to convert biomass derived carbohydrates to hydrocarbon fuels. Recently, Bond, et al., reported a strategy by which aqueous solutions of γ-valerolactone (GVL), produced from biomass-derived carbohydrates, can be converted to liquid alkenes in the molecular weight range appropriate for transportation fuels by an integrated catalytic system that does not require an external source of hydrogen (Bond, 2010). In the first step, butene is produced from γ-valerolactone via decarboxylation over a silica-alumina catalyst. In the second step, the butene formed undergoes oligomerization over an acid catalyst such as H-ZSM-5 to form gasoline and/or jet fuel range alkenes. In another effort, Gürbüz, et al., upgraded mono-functional intermediates produced by catalytic conversion of sugars and polyols over Pt—Re/C catalysts (consisting of alcohols, ketones, carboxylic acids, and heterocyclic compounds) to fuel-grade compounds using two catalytic reactors operated in a cascade mode (Gürbüz, 2010). These intermediates were further upgraded to hydrocarbon fuels using two different catalytic reactors consisting of three different catalysts (CeZrOₓ and Pd/ZrO₂ in the first reactor and Pt/SiO₂—Al₂O₃ in the second reactor). Li and Huber (2009) reported sorbitol hydrodeoxygenation over a Pt/SiO₂—Al₂O₃ catalyst below 250° C. and at 450 psig. The reaction produced several oxygenates (alcohols, ketones, and cyclic ethers) in both liquid and vapor phases. It was proposed that a number of reactions including C—C bond cleavage, C—O bond cleavage, dehydration, and hydrogenation occur during sorbitol hydrodeoxygenation resulting in the observed products. These processes have capital and operating costs that may be impractical due to multiple steps and expensive catalysts involved in the biomass conversion.

Sughrue, et al., US-2011-0046423, hydrotreat a mixture of sorbitol and diesel over a commercial hydrotreating catalyst to produce lighter alkanes and hexanes desirable for gasoline fuels. Lotero, et al., US-2011-0144396, provide a process comprising steps of a) providing a biomass feedstock; b) de-oxygenating the biomass feedstock to form a solid-intermediate; and c) liquefying the solid-intermediate to produce a biocrude. Yao, et al., US-2011-0087060, mitigate potential coking and to moderate the temperature of the catalyst bed while maintaining high conversion of sugar alcohol to hydrocarbon via a hydrotreating process, a diesel feedstock is fed over the reactor catalyst with multiple injections of polyol feedstock along the reactor. Yao, et al., US-2011-0152513, provide a process for the conversion of carbohydrates and polyols to hydrocarbons in which the rate of coke formation and the production of COₓ by-products during the conversion is minimized. Jess, et al., US-2011-0184215, improve biomass pyrolysis where the heat source is a hot petroleum feedstock, which provides heat and may also contribute organic material to the pyrolysis reaction. Anand, et al., U.S. Ser. No. 13/233,256 filed Sep. 15, 2011, entitled “MoS₂ CATALYST FOR THE CONVERSION OF SUGAR ALCOHOL TO HYDROCARBONS,” developed a sulfur-tolerant methanation catalyst and a sulfur-tolerant methanation process.

What is desired is an efficient reaction to convert biomass and biomass byproducts that requires a minimum number of reactors and produces a high yield of hydrocarbon products useful as fuels. It would certainly be preferred if such processes produced increased distillate range fuels rather than lighter fraction hydrocarbons which are more challenging to blend into gasoline because of vapor pressure as well as other reasons.

### BRIEF SUMMARY OF THE DISCLOSURE

The invention more particularly includes a process for producing longer carbon chain hydrocarbon products from shorter carbon chain oxygenate material by contacting hydrogen and the oxygenate material with a hydrocondensation catalyst which catalyzes the formation of carbon-carbon bonds in the presence of hydrogen to form longer chain hydrocarbon products.

The invention also relates to a system for converting biomass oxygenates to renewable C5-C25 hydrocarbons wherein the system includes an oxygenate feedstock derived at least in part from biomass and comprising one or more polyols, sugars, or carbohydrates in an aqueous solution along with a hydrogen feed. A hydrocondensation reactor including a hydrocondensation catalyst is included for converting the oxygenate feedstock into longer chain hydrocarbons and a distillation column is arranged for separating the renewable hydrocarbons into product streams comprising C5-C25 range hydrocarbons comprising renewable naphtha, renewable gas oil, and at least one distillate range renewable fuel.

### BRIEF DESCRIPTION OF THE DRAWINGS

A more complete understanding of the present invention and benefits thereof may be acquired by referring to the following description taken in conjunction with the accompanying drawings in which:

FIG. 1 shows a schematic diagram of the system for producing fuels directly from biomass oxygenates;

FIG. 2 shows a schematic diagram of an alternative embodiment of the system for producing fuels directly from biomass oxygenates;

FIG. 3 shows a schematic diagram of a second alternative embodiment of the system for producing fuels directly from biomass oxygenates;

FIG. 4 shows a chart of the performance of PtPd catalyst for sorbitol hydrotreating. Conversions reported based on total organic carbon data (1,200 psig, WHSV=0.16 g sorbitol/g cat/h);

FIG. 5 shows a chart of the distribution of inlet carbon (as sorbitol) into vapor, organic, and aqueous phases as a function of temperature;

FIG. 6 shows a chart of the carbon number distribution of the products from the hydrocondensation plus hydrodeoxygenation process;

FIG. 7 shows a hydrocarbon type distribution from early tests using hydrocondensation in combination with hydrodeoxygenation to make longer chain hydrocarbons;

FIG. 7A shows a hydrocarbon type distribution from later tests using hydrocondensation in combination with hydrodeoxygenation to make longer chain hydrocarbons where more naphthenes and less paraffins are produced;

FIG. 8 shows a chart of the time-on-stream behavior of hydrocondensation of sorbitol at 270° C. and 340° C.;

FIG. 9 shows a diagram of the fixed bed reactor used in the lab;

FIG. 10 shows a chart of deactivation of a hydrocondensation catalyst as a function of time-on-stream. Xa is the carbon conversion obtained from the Total Organic Carbon method;

FIG. 11 shows a chart comparing boiling point curves of an intermediate product (pre-polishing or pre-hydrodeoxygenation) and the final hydrocarbon product obtained after polishing or after hydrodeoxygenation;

FIG. 12 is a chart that shows the distribution of inlet carbon to various hydrocarbon and carbon oxides;

FIG. 13 shows a chart indicating the variation in sugar alcohol conversion (obtained from HPLC) as a function of TOS;

FIG. 14 is a chart showing the plot of product yield versus 1^{st} bed (PdPt on Silica/Alumina catalyst) weight hourly space velocity. Maxima are observed near 1 h⁻¹ and above 1.6 h⁻¹; and

FIG. 15 is a chart showing that the products of the process are distillable into fractions. About 70 percent boils off at about 300 degrees Fahrenheit and the last 30 percent that boils off at higher temperatures shows that longer chain hydrocarbons are being formed by the process.

### DETAILED DESCRIPTION

Turning now to the detailed description of the preferred arrangement or arrangements of the present invention, it should be understood that the inventive features and concepts may be manifested in other arrangements and that the scope of the invention is not limited to the embodiments described or illustrated. The scope of the invention is intended only to be limited by the scope of the claims that follow.

Abbreviations used herein include flame ionization detector (FID), gas chromatograph (GC), hydrodeoxygenation (HDO), hydroprocessing (HPC), high-pressure liquid chromatography (HPLC), ignition quality tester (IQT), nitric oxide ionization spectroscopy evaluation (NOISE), outside diameter (OD), standard cubic centimeter per minute (sccm), simulated distillation (SIMDIS), total acid number (TAN), thermal conductivity detector (TCD), total organic carbon (TOC), time-on-stream (TOS), ultra low sulfur diesel (ULSD), and weight hourly space velocity (WHSV).

Currently, hydrocarbon fuels derived from mineral sources such as crude oil and natural gas, etc. are less costly to produce than fuels derived from renewable or biomass sources. Mineral hydrocarbons have very high energy density and are found in relatively large deposits so transportation to a refinery is far less complicated and costly as compared to handling and transporting raw biomass. Moreover, transportation of feedstocks by large pipelines permits conventional refineries to enjoy an economy of scale that cannot be matched by currently envisioned commercial scale biomass refineries. Thus, it is generally recognized that profitable biomass refineries will have to be simple and efficient to be cost competitive as long as mineral sourced hydrocarbons are viable and available. “Simple”, in this context, basically means few reactors. “Efficient” generally means high productivity and low capital and operating costs including low catalyst cost.

The present invention includes a process for converting biomass-derived sugar alcohols and hydrogen directly into a mixture of oxygenates and hydrocarbons via a hydrocondensation reaction in a hydrocondensation reactor. This mixture of oxygenates and hydrocarbons, upon a further step of hydrodeoxygenation, produces a fungible and distillable hydrocarbon product boiling in the range of 50° F.-1000° F. which generally translates into a C5-C25 product slate. This product consists of light naphtha, heavy naphtha, jet, diesel, and gas oil fractions and their boiling ranges and corresponding volumetric yields are shown in Table I. As these fuels are derived from biomass with an expected 50% reduction in greenhouse gas emissions compared to petroleum-derived fuels, production of these fuels will earn credits under the Renewable Fuel Standard 2 (RFS2) of the US Energy Independence and Security Act of 2007 and therefore have value in addition to their basic value as fuels or other commodities. It is believed that each fraction of these biofuels from distillation will be fungible and relatively easily sold to customers for value. It should be noted that with feedstocks having 5 or 6 carbon atoms in the carbon chains and hydrocarbon products having carbon chains of greater than 6 carbon chains indicates that the feedstock is being converted to longer chain hydrocarbons. Although, not every molecule in the feedstock is converted to a longer chain hydrocarbon, some considerable portion of the feedstock is converted to longer chain hydrocarbons and this provides an advantage in delivering products that are in demand and especially if the products are drop in fuels meeting current fuel standards and may be blended with other on-spec fuels without concern for the resulting blend to become non-conforming to the specifications to the fuel.

It has been recognized that more than sugar alcohols may be converted by the present invention wherein the feed may be described as an oxygenate material. Some of the oxygenate material may have a relatively significant ratio of oxygen to carbon. Sugar alcohols such as xylitol and sorbitol have one oxygen for every carbon. Other oxygenate materials suitable for the invention have less. It is expected that oxygenate materials having an average of at least 0.6 oxygens for each carbon may be desirable for creating a financially viable process. Higher oxygen content, such as at least 0.65 oxygens for each carbon, at least 0.70 to one, at least 0.75 to one and at least 0.8 to one are increasingly attractive.

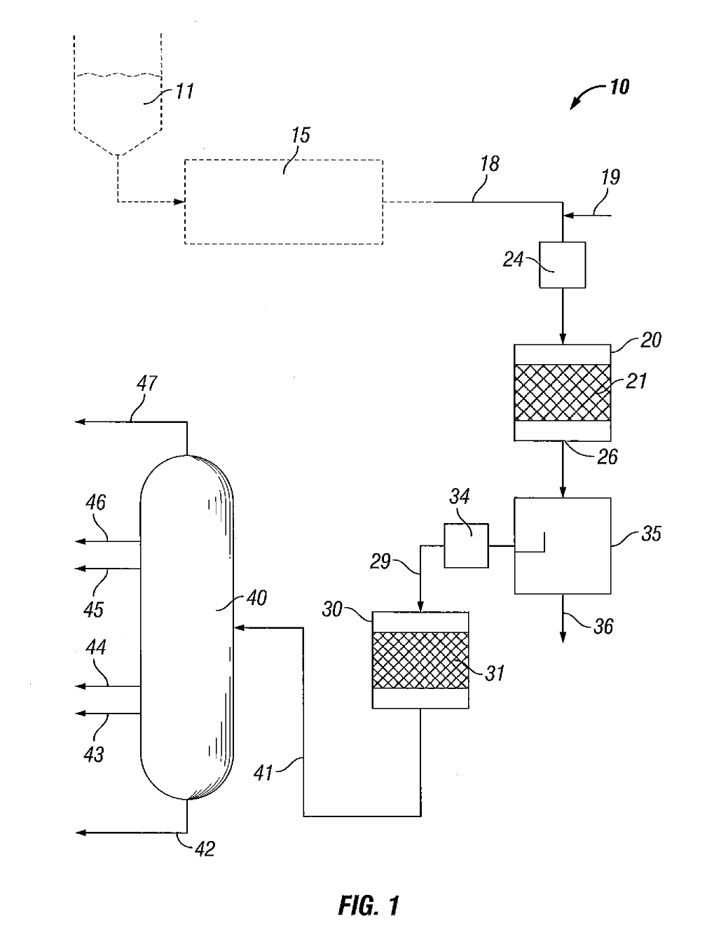

FIG. 1 shows a system 10 for the conversion of oxygenate material or materials to hydrocarbons where the conversion occurs in one step in conversion reactor 20. The-oxygenate material is supplied to the reactor 20 in an aqueous solution via an oxygenate supply 18. The oxygenate material is derived from biomass, but the conversion of biomass to oxygenate material may occur at a distant location from the site of system 10. In that case the oxygenate material is transported to the oxygenate conversion system 10. However, the biomass itself may be transported to a location adjacent the system 10 and converted to oxygenate material at the same site. As shown in dotted lines, whether at the oxygenate conversion system 10 or remotely, the biomass is conceptually supplied in hopper 11 to a biomass conversion process 15. The biomass and more particularly, the cellulose and hemicellulose of the biomass are converted to the oxygenate material in the biomass conversion process 15 and delivered via the oxygenate material supply 18. Oxygenate supply 18 may include one or more storage vessels. The biomass conversion process 15 may include a number of reactors and steps. Again, efficiency will suggest a simple and productive biomass conversion process 15 where oxygenate conversion system 10 is able to convert much of the feedstock to hydrocarbons.

At the center of the oxygenate conversion system 10 is a hydrocondensation reactor 20 including a fixed hydrocondensation catalyst bed 21. Hydrogen is supplied via hydrogen feedline 19. Suitable known equipment associated with feeding the oxygenate material and the hydrogen at desired temperature and pressure conditions, as is known in the art, is shown as condition controlling mechanism 24. The products exiting the reactor at outlet 26 include an aqueous phase and an organic phase. While the oxygenate material is provided in water, once the hydrocarbons are formed in the reactor 20, they tend to separate from the aqueous phase and may be gravity separated in phase separator 35. Such separators are well known. In the first embodiment, the products at the outlet 26 are separated in separator 35 into an organic phase that is directed to a hydroprocessing step in reactor 30 or more preferably a hydrodeoxygenation process in reactor 30. The reactor 30 preferably includes a fixed bed of hydrodeoxygenation catalyst 31 and includes a hydrogen feedline 29. The hydrodeoxygenation reactor 30 is operated under controlled temperature, pressure and rate established by hydroprocessing condition controlling mechanism 34

So, once the organic phase is separated from the aqueous phase in phase separator 35 and hydrodeoxygenated in reactor 30, the organic phase is directed to a distillation column 40 via line 41 where it is separated into separate boiling fractions. Distillation columns are well known technology for separating crude oil into fuel fractions such as gasoline and diesel. In the present invention, the organic products separate quite well into light naphtha, heavy naphtha, jet, diesel and gas oil. The heaviest component is the gas oil which comes out near the bottom at gas oil outlet 42. Diesel fuel is the next heaviest and comes out at diesel outlet 43 while jet comes out at jet outlet 44. Heavy naphtha comes out at heavy naphtha outlet 45 and light naphtha comes out at light naphtha outlet 46. If any gas is formed in the system, it exits at the top of the distillation column 40 through gas outlet 47.

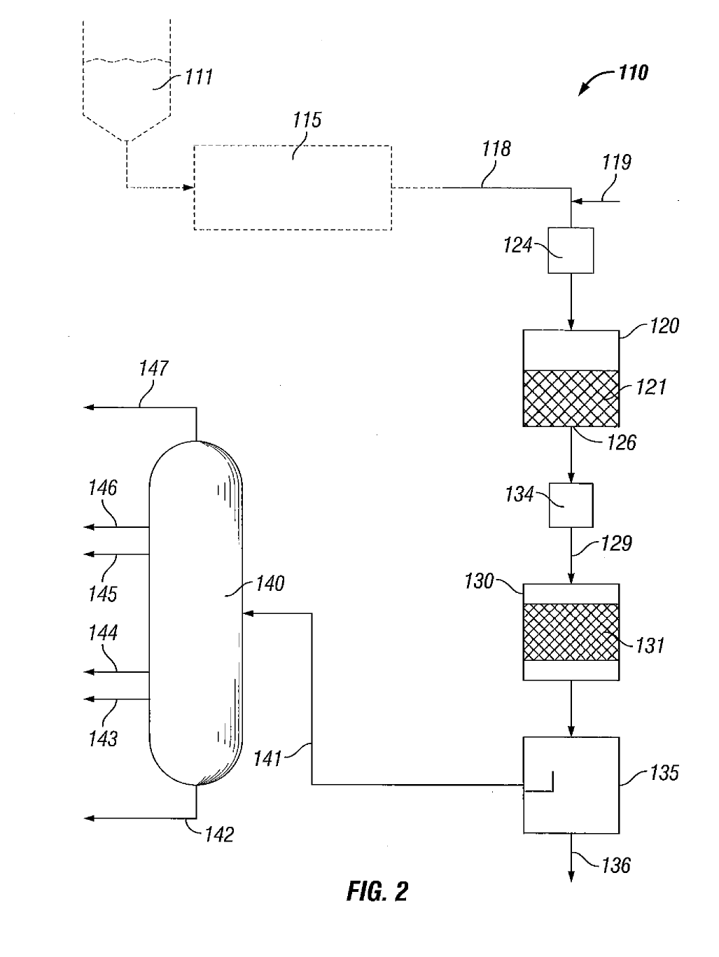

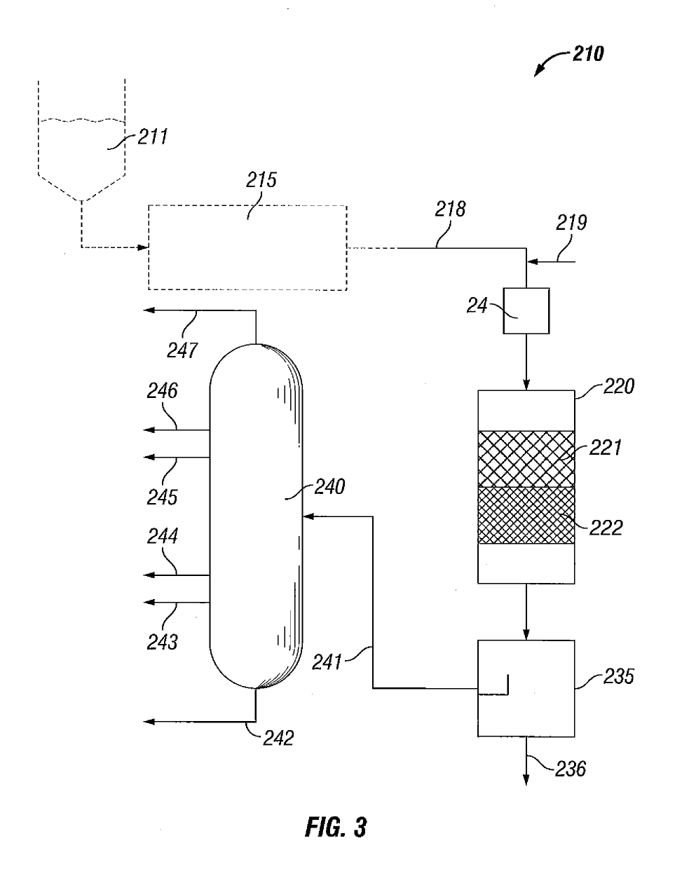

Two alternative embodiments of the oxygenate conversion system 10 are also shown in FIGS. 2 and 3 where similar elements are similarly numbered but with the addition of “100” or “200” to the reference number. So, for example, in FIG. 2, the oxygenate conversion reactor is indicated by the reference number 120 and in FIG. 3 by the reference number 220.

Turning to FIG. 2, the oxygenate conversion system 110 is quite similar to the system 10, but one difference is that the entire product stream exiting the hydrocondensation reactor 120 is delivered to the hydrodeoxygenation reactor 130. Although the hydrodeoxygenation reactor 130 must be bigger to handle the additional volume of liquid, by removing oxygen from organic material in the product stream, some molecules may have shifted to favor the organic stream as compared to the aqueous phase. The tradeoff is a larger reactor and larger catalyst bed and all the associated fixed and operating costs to acquire more preferred products going into the distillation tower 140 and coming out in the fuel fractions.

In FIG. 3, some process efficiency was sought by eliminating the separate reactor vessel for the hydrodeoxygenation step by including the hydrodeoxygenation catalyst in the hydrocondensation reactor 220. This embodiment would have a similar advantage as the FIG. 2 embodiment without having a second separate reactor vessel.

In some early tests with system 10, it was found to be desirable to provide an additional feedstream of diesel or other middle distillate range hydrocarbon to reduce coking However, now, it has been found that eliminating such a feedstream actually improves the product selectivity toward longer chain hydrocarbons and it is preferred not to co-feed a diesel range hydrocarbon with the oxygenate material. There may be options to include other co-feeds with the oxygenate material for temperature control or other reasons, but to the extent that such co-feeds might include hydrocarbons, they will be C14- and perhaps even lighter such as C10-hydrocarbons or even as light as hexane. However, while it is believed to be disadvantageous to co-feed heavy hydrocarbons (C10+) with the oxygenate, injecting hydrocarbons such as naphtha, jet, diesel or gas oil for temperature control at various locations along the reactor may provide an efficient and effective method for reactor temperature control. Such hydrocarbons injected downstream in the reactor tend not to interfere with distillate selectivity or productivity of longer chain hydrocarbons. Temperature control may also include recycling products from the hydrocondensation reactor.

In the system 10 of the present invention, it is seen that it is relatively simple, while produces a range of hydrocarbon products that includes a higher percentage of naphthenes or cycloalkanes in its product slate. These are fully saturated hydrocarbons that comprise one, two or three rings often with side chains. Naphthenes are very attractive for diesel and jet fuels as naphthenes with requisite portions of normal paraffins and isoparaffins provides middle distillate fuels with high cetane ratings (which is like octane to gasoline) that meet specifications and are also free flowing liquids at very cold temperatures. To the extent that other biomass conversion systems are available to produce hydrocarbons from biomass, such systems produce fuels with more light hydrocarbons, less naphthenes and considerably more aromatics as compared to the distillate fuel products of the present invention. High molecular weight paraffins provide high cetane, but have poor cold flow properties. Aromatics are generally not desirable in higher concentrations for jet and diesel (above 25 weight percent exceeds specifications). A combination of paraffins with higher naphthene content seems to provide a very attractive distillate fuel or fuel blendstock. To provide an understanding of the fuels created by this simple oxygenate conversion system, the fractions are shown below in Table I along with their general boiling range and a simple projection of the volumetric yield of each fuel.

TABLE I
Basic Fractions

| | Boiling | | |
|---|---|---|---|
| | range | Volumetric yield | Potential market |
| Fraction | (° F.) | (%) | destinations |
| Light | 50-170 | 50 | Gasoline blendstock |
| naphtha | | | Chemicals and |
| | | | Solvents |
| Heavy | 170-310 | 15 | Gasoline blendstock |
| naphtha | | | |
| Jet | 310-565 | 15 (some amounts of this | Drop-in |
| | | volume may be shifted into | |
| | | the diesel product) | |
| Diesel | 310-680 | 30 (note that up to half of | Drop-in |
| | | this volume may be | |
| | | directed into jet product) | |
| Gas oil | 680-1000 | 5 | FCC feedstock |
| | | | Hydrocracker |
| | | | feedstock |
| | | | Residual marine fuel |
| | | | blendstock |

The volumetric yields show that about half of the organic fraction is light naphtha. While the product slate would be more valuable if it could be shifted to more products that are as heavy as jet, diesel or gas oil, volume measurements tend to understate the ratio of carbon in the heavier fractions. Also, to the extent that about half of the products are heavy naphtha and heavier or that about 30% is jet and diesel appears to be a big step toward a desirable result as compared to most bio-sourced materials ending up in light naphtha or hydrocarbon gases. Getting 30% of the products into jet and diesel in a single step conversion is a notable advantage of the present system.

It should be noted that current plans for bio-sourced fuels is to blend it with petroleum sourced fuels up to a maximum of 50% bio-sourced fuel in the final fuel delivered to the consumer. As such, the characteristics of the consumer fuel will be influenced no more than half by the bio-sourced fuel. So, depending on the naphthene content of the petroleum component of the final fuel, the high naphthene content in the bio-sourced fuel may be diluted by at least fifty percent by the petroleum component of the final fuel and maybe more.

As noted above, distillate fuels are likely to be the most attractive hydrocarbon products simply because these products currently hold higher prices in the market place on a weight basis. Jet fuel has very detailed and stringent standards, but is comprised of hydrocarbon molecules having carbon chains where the molecules have between about 8 or 9 up to about 16 carbons each. Commercial airline jets in the US use Jet A, while internationally the standard is Jet A1. The US military has its own standard designated as JP-8. Diesel overlaps with jet fuel at the low end of the diesel fuel fraction and is also saleable if it meets a very detailed specification, generally known as Number 2 Diesel. Diesel is comprised of hydrocarbon molecules with carbon chains where the total number of carbons in the molecules numbers between about 8 and 21 carbons for each molecule.

In addition to producing more attractive distillate fuels, it is believed that gas oil from the present invention, sometimes also called fuel oil, will also be attractive compared to conventional gas oils. Gas oil is a heavier fraction than diesel having up to about 25 carbons, and it is believed that high naphthene content in gas oil will be at least as attractive as conventional gas oil and potentially more attractive such as for its cold flow properties. In general, “attractive” suggests that there may be a price premium for the product, although the price premium may be small and variable. As with any refinery, it is quite important that the products of the refinery are saleable, even if some products are quite discounted, as long as the return for the full range of products exceeds the costs. But having unsalable products that must be disposed at loss, especially if very costly to dispose, is very, very unattractive.

The last fraction, but actually the lightest fraction is naphtha. Full range naphtha consists of a mixture of hydrocarbon molecules generally having between about 5 up to about 10 sometimes up to 12 carbon atoms. Light naphtha typically consists of molecules with 5-6 and maybe 7 carbon atoms. Heavy naphtha consists of molecules with 6 or 7 carbons up to 10 to 12 carbons. Naphtha is preferably used as feedstock for high octane gasoline although it has other uses such as for producing olefins in steam crackers, and as a solvent. In the present invention, the production of naphtha appears to fit with currently marketed and sold naphtha so it is believed that naphtha production will be readily marketed and sold.

Turning back to the process, biomass that is converted to oxygenate material for the invention includes a variety of feedstocks comprising oxygenates. Biomass may be derived from any biological material that contains sugars, carbohydrates, lignins, fatty acids, proteins, oils, and other components. Biomass may include materials from forest residues (such as dead trees, branches, leaves and tree stumps), yard clippings, wood chips, wood fiber, corn fiber, sugar beets, sugar cane, corn syrup, algal cultures, bacterial cultures, fermentation cultures, and the like. In one embodiment, biomass is derived from waste products and low value residues remaining after other processes such as paper manufacturing waste, farming residues, food manufacturing waste, meat processing waste, municipal solid waste, animal waste, biological waste, and sewage. In another embodiment, biomass is derived from plant materials such as miscanthus, switchgrass, hemp, corn, poplar, willow, sorghum, sugarcane, and a variety of tree species, ranging from eucalyptus to oil palm (palm oil). Oxygenates may be generated from biomass through solubilization, acid hydrolysis, pyrolysis, and other liquefaction methods used to convert solid biomass and large molecules to smaller aqueous and organic liquids.

While there is substantial diversity of biomass that may be converted to oxygenate material for the present invention, there is also quite a wide variety of oxygenate materials that may then be used as feedstock the present invention. The oxygenate material provided by the biomass conversion process 15 may potentially comprise oxygenates including carbohydrates, sugars, pentoses, hexoses, monosaccharides, dextrose, glucose, α-D-glucopyranose, β-D-glucopyranose, α-D-glucofuranose, β-D-glucofuranose, galactose, disaccharides, levoglucosan, sucrose, manose, xylose, isosorbide, lactose, maltose, fructose, cellobiose, melibiose, raffinose, glyceraldehyde, erythritol, xylitol, sorbitol, arabitol, mannitol, dulcitol, maltitol, arabinitol, isosorbide, glycerol, glycerin, alcohol, methanol (MeOH), ethanol (EtOH), isopropyl alcohol (IPA), butanol (BuOH), n-butanol, t-butanol, ethers, methyl tert-butyl ether (MTBE), tertiary amyl methyl ether (TAME), tertiary hexyl methyl ether (THEME), ethyl tertiary butyl ether (ETBE), tertiary amyl ethyl ether (TAEE), diisopropyl ether (DIPE), hydroxymethyl-tetrahydrofuran or tetrahydro-2-furfuryl alcohol (THFA), methyl-tetrahydrofuran, 2-methyltetrahydrofuran, 3-methyltetrahydrofuran, tetrahydrofuran, diols, methanediol (H₂C(OH)₂), ethylene glycol, propane diols, 1,2-propanediol, 1,3-propanediol, butanediols, 1,2-butanediol, 1,3-butanediol, 1,4-butanediol, 2,3-butanediol, pentane diols, 1,2-pentanediol, 1,5-pentanediol, octanediol, 1,8-octanediol, etohexadiol, p-menthane-3,8-diol, 2-methyl-2,4-pentanediol, aldehydes, propanal, butanal, 2,5-furan-dicarboxyaldehyde, carboxylates, acetic acid, oxopropanoic acid, acrylic acid, levulinic acid, succinic acid, 2,5-furan-dicarboxylic acid, aspartic acid, glucaric acid, glutamic acid, itaconic acid, acetylacrylic acid, 4-O-Me-glucuronic acid, gluconic acid, xylonic acid, esters, levuninate esters, lactones, valero lactone, α-methylene-γ-valerolactone, angelilactones, trisaccharides, oligosaccharides, polysaccharides, starch, and the like including derivatives, dimers, trimers, and polymers. Polyols include glycerol, sorbitol, xylitol, and the like. Oxygenate feedstocks consist of one or more oxygenates in an aqueous solution. Liquefaction of biomass typically produces feedstocks containing sorbitol and xylitol. Oxygenate feedstocks consist of one or more oxygenates in an aqueous solution. Feedstocks may contain from about 50 to about 98% v/v oxygenates. In one embodiment an oxygenate feedstock contains between 20% up to 98% sorbitol, xylitol and mixtures of sorbitol and xylitol. Although sorbitol feedstock comprises sorbitol and aqueous solution, additional oxygenates, polyols, oils, and sugars are present after liquefaction. Many isomers, polymers, and soluble sugars are present in the aqueous liquefaction fraction. Hydrotreating will convert many of these to valuable fuel products. Preferred oxygenate feedstocks to reactor 20 are sugar alcohols, sugars, sugar derivatives, hydrogenated sugars, hydrogenated sugar derivatives, glycerol, tetrahydrofurfuryl alcohol, isosorbide, sorbitans, and C3 to C6 polyols and any combination thereof.

The hydrocondensation catalyst in catalyst bed 21 may be selected from a variety of materials including noble metal catalysts on a support or various supports, promoted noble metal catalysts, including specific noble metal catalysts like platinum-palladium (Pt—Pd) catalysts, germanium-containing zeolite catalyst, nickel-tungsten (Ni—W), and the like, or catalysts containing oxidation resistant noble metals from groups VIIb, VIII, and Ib of the second and third transition series, including rhenium, ruthenium, rhodium, palladium, silver, osmium, iridium, platinum, gold and the like. Other oxidation resistant metals include mercury, titanium, niobium, tantalum, tungsten, and the like. Noble metal aromatization catalysts are available from a variety of commercial producers including AKZO-NOBEL®, ALBEMARLE®, AXENS, GENTAS, HALDOR TOPSØE AS, Johnson Matthey, W.R. GRACE & CO., which produce many hydrotreating catalysts like the HALDOR TOPSØE TK-335, TK-339, TK-341, and TK-351, Johnson Matthey PRICAT PD and PT/Alumina, KETJENFINE® (KF) 200-A, ALBEMARLE® KF-200 and KF-201, AXENS LD catalyst family, Grace-Davison ALCYON™, and similar catalysts. Noble metal catalysts may also be synthesized as described in 056013173, US6884340, US6872300, and the like. Other noble metal catalysts may be purchased or synthesized either as single metal or bi-metal catalysts including Pt/SiO₂—Al₂O₃, PtPd/SiO₂—Al₂O₃, Pd/SiO₂—Al₂O₃, Pt/SiO₂, PtPd/SiO₂, Pd/SiO₂, Pt/Al₂O₃, PtPd/Al₂O₃, Pd/Al₂O₃, Pt/Zirconia, PtPd/Zirconia, Pd/Zirconia, and the like. It has also been found that base metals will work as hydrocondensation catalysts including Ni, Mo, Co, W and combinations thereof including bimetallic catalysts. These catalysts may be supported on Al₂O₃, SiO₂, zeolite, or other support.

The hydrodeoxygenation catalyst is typically a base metal catalyst and there are a variety of available catalysts that comprise Ni, Mo, Co, W and combinations thereof the like on Al₂O₃, SiO₂, zeolite, or other support. Hydrodeoxygenation catalysts may contain metals and combinations of metals with molybdenum, tungsten, cobalt, or nickel. Hydrodeoxygenation catalysts are commercially available from a variety of sources including BASF Ni catalyst, NIPPON KETJEN Co. like the KF, KG, KFR and KAS catalysts, AXENS HR catalyst family, HALDOR TOPSØE AS like the TK catalyst family, ALBEMARLE®, W.R. GRACE & CO., AXENS, GENTAS, and others. Refining catalysts are also readily available from a variety of other sources including ADVANCED REFINING TECHNOLOGIES (ART), AMERICAN ELEMENTS, EURECAT, FISCHER, HEADWATER, Johnson Matthey, PGM CATALYSTS & CHEMICALS, SIGMA, and other chemical suppliers. Catalysts may be supported on an alumina, silica, titania, zeolite, carbon, plastics, ceramics, or other support materials. Catalysts may be microsized, nanosized, fluidized or other catalyst forms dependent upon the reactor size, shape and conditions under which the reaction is run.

### EXAMPLES

All gases described herein are commercially available and may be purchased from a variety of suppliers. Unless otherwise specified, gases used were ultra-high purity gases from AIRGAS®.

### Example 1

### Oxygenate Hydrocondensation

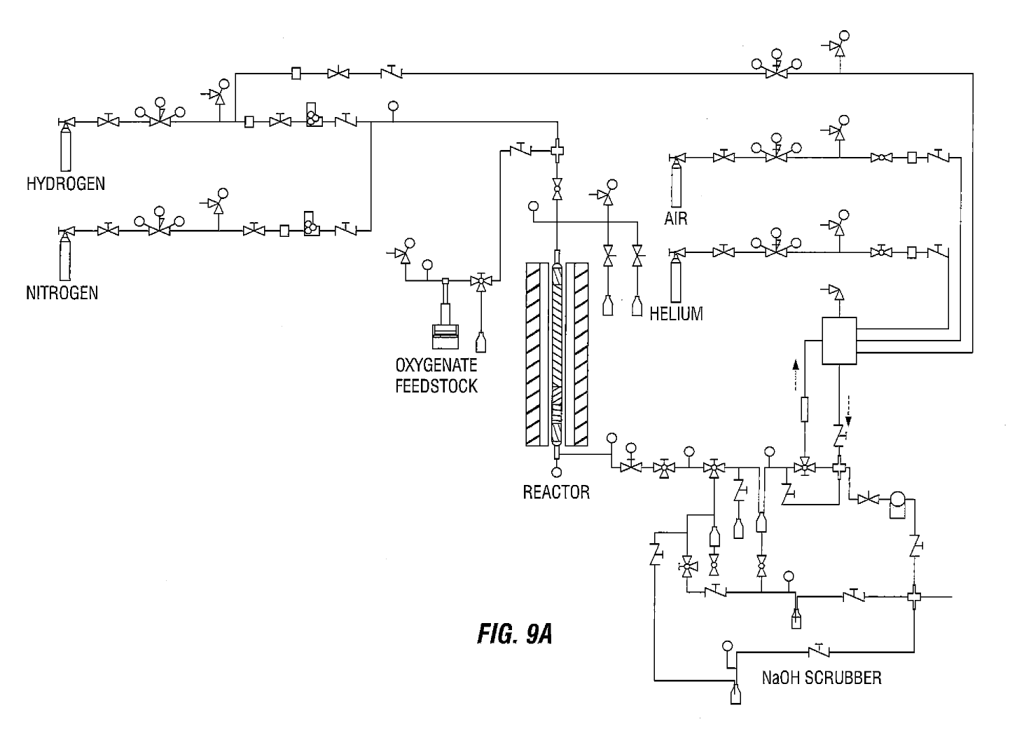

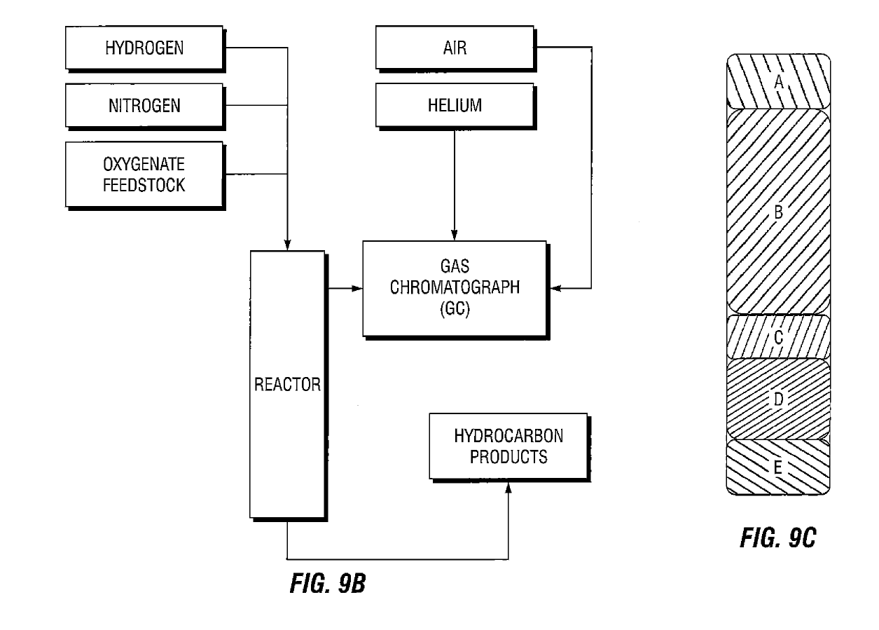

In one embodiment, a silica-alumina supported platinum-palladium hydrocondensation catalyst (Pt/Pd catalyst) was used to convert oxygenates to hydrocarbon fuels. A 70 wt % sorbitol in water mixture was diluted to 40 wt % sorbitol using distilled water. To ensure a safe operation, a ventilated enclosure encased the entire fixed-bed reactor (FIG. 9). Because sorbitol hydrotreating reaction required 1,200 psig, the reactor setup had several safety features. All gas cylinders and the reactor had pressure relief valves set at 1,450 psig. The ISCO™ syringe pump had an in-built pressure control system to cease pumping when the pressure exceeded 1,400 psig. The pump also had a pressure relief valve set at 1,450 psig. The reactor furnace controller had an override set at 500° C. Handling of all catalyst samples and separation of collected liquid products was conducted in a ventilated hood.

The model biocrude hydrotreating was conducted in a fixed-bed reactor system (FIG. 9). A platinum-palladium noble metal catalyst extrudate diluted with alundum was packed in a ¾″ OD reactor. The catalyst was reduced in the presence of hydrogen by following a standard reduction procedure. Briefly, the reactor temperature was increased from room temperature to 120° C. at 2° C./min and held at 120° C. for 2 h to remove moisture. The hydrogen flow was 100 Nm³/m³ cat/h. The pressure was increased to 145 psig, and the temperature was increased to 350° C. at 0.3° C./min, then to 450° C. at 0.2° C./min. The catalyst was reduced at 450° C. for 16 h. Following reduction, the temperature was decreased to 50° C. at 2° C./min.

After reduction, the catalyst was wetted with the sorbitol-water feed introduced using an ISCO™ syringe pump for 2 hours at a liquid hourly space velocity of 3 h⁻¹. Following this step, the reactor temperature was increased to the desired value, and the pressure was increased to 1,200 psig. The reaction feed consisted of a 40 wt % sorbitol solution in water and 250 sccm of hydrogen. No diesel was co-fed with sorbitol in these experiments. The weight hourly space velocity used in all experiments was 0.16 g sorbitol/g cat/h. For each data point, products were collected for at least 24 h to achieve constant conversions.

The off-gases from the reactor were analyzed using an AGILENT® 6890 GC equipped with two detectors (TCD and FID) and two columns (a CARBOXEN™ column for permanent gases and HP-1 column for the hydrocarbons).

The liquid products collected were split into organic and aqueous phases by gravity separation. The aqueous phase was analyzed for unreacted sorbitol and intermediate oxygenates by the total organic carbon (TOC) method and by HPLC.

The organic phase was analyzed using an AGILENT® 7890 GC equipped with FID and a HP-1 column for hydrocarbon analysis. For detailed characterization, the organic product was analyzed using GC-MS TOF, GC-Atomic Emission Detector (AED), Detailed Hydrocarbon Analysis (DHA), simulated distillation by ASTM D 2887 (SIMDIS), Karl-Fischer titration for water, and combustion for elemental analysis.

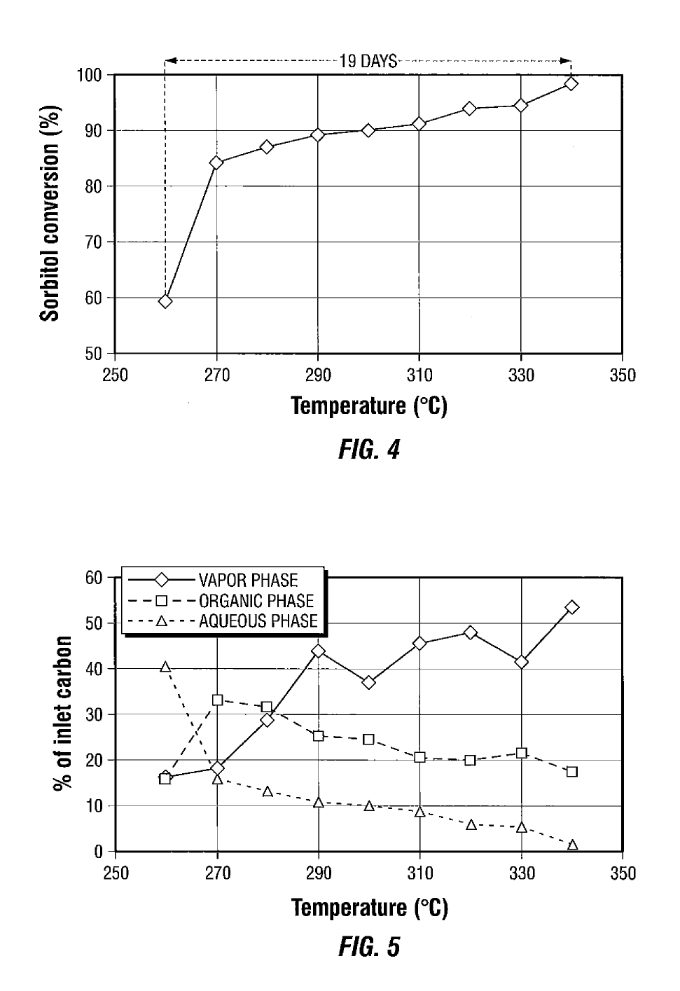

Above 260° C., the carbon conversion calculated by total organic carbon (TOC) method increased significantly with temperature, as shown in FIG. 4. About 60% conversion was achieved at 260° C., and the value increased to 98% at 340° C. At all data points, the mass balance was >92%. The product darkened from pale yellow to yellow on exposure to room light and air. Products collected at higher temperatures did not show any coloration. GC analysis indicated the presence of hydrocarbons larger than hexane in both liquid and vapor phases. The overall C6+ selectivity (hexane and hydrocarbons heavier than hexane) was 60-70%, C5−selectivity was 25-35%, and CO₂ selectivity was 5%.

Carbon distribution between aqueous, organic, and vapor phases as a function of temperature is shown in FIG. 5. From the figure, it is observed that at 260° C., almost 40% of the inlet carbon (as sorbitol) was in the aqueous phase while about 30% was in organic and vapor phases. With increasing temperature, the carbon in the aqueous phase decreased, and the carbon in vapor phase increased. The carbon in the organic phase showed a maximum at 270° C. The carbon distribution values in vapor phase are underestimated due to the lack of GC capability to completely analyze vapor phase products. Overall, >80% of inlet carbon was converted to organic molecules in liquid and vapor phases at 340° C.

The liquid products from Example 1 are separated into an aqueous fraction and an organic fraction. The organic fraction is subjected to a separation based on boiling fractions in a distillation tower into the five fractions described above

The density of all organic samples was between 0.65-0.95 g/cc. Oxygen content was measured using elemental analysis, GC-Atomic Emission Detector (GC-AED), and Karl-Fischer titration. Elemental analysis and GC-AED indicated the concentration of oxygen in the organic phase decreased from an inlet value of 52 wt % to 23 wt % at 260° C. and 6.5 wt % at 340° C. Of the 23 wt % oxygen remaining after reaction at 260° C., 17 wt % was oxygenates (indicated by GC-AED) and 5 wt % was dissolved water in the organic phase (indicated by Karl-Fischer titration). At 340° C., oxygenates were the main source of oxygen as the concentration of oxygen from water was 0.1 wt % (see Table II).

TABLE II
Temperature dependence of oxygen concentration in the organic
phase of the hydrocondensation conversion calculated using GC-
AED, Karl-Fischer titration (for water), and combustion method.

| Temperature | O from | O from Karl-Fisher | O from Combustion |
|---|---|---|---|
| (° C.) | AED (wt %) | titration (wt %) | Method (wt %) |
| 260 | 17 | 4.2 | 23 |
| 340 | 2.5 | 0.1 | 6.5 |

Organic products collected for Example 1 at 260° C., 270° C., and 340° C. were analyzed by GC-MS to identify the oxygenates and hydrocarbons. Table III lists oxygenates and Table IV lists hydrocarbons present at concentrations above 0.4 wt %.

TABLE III
Oxygenates present in organic products at 260°
C., 270° C., and 340° C.

| | 260° C. | 270° C. | 340° C. |
|---|---|---|---|
| Compound | (wt %) | (wt %) | (wt %) |
| 1-butanol | 1.2 | — | — |
| c/t-2,5-dimethyl-THF | 2.0 | 0.5 | — |
| 2-methyltetrahydropyran | 3.0 | 1.3 | — |
| 3-methyltetrahydropyran | 8.0 | 4.5 | 1.2 |
| 1-pentanol | 2.5 | 1.5 | — |
| 3-hexanone | 11.9 | 7.8 | 1.5 |
| 2-hexanone | 4.8 | 2.2 | 0.8 |
| 1-hexanol | 21.3 | 15.1 | — |
| Hexanoic acid | 2.4 | 3.7 | — |

Oxygenate content measured by GC-MS.

Oxygenates dominated at low temperature with hexanol being the most abundant product (22 wt %) at 260° C. followed by hexanone (17 wt %), methyl tetrahydropyran (11 wt %), pentanol and its derivatives (6 wt %). The hydrocarbons consisted of C6-C18 n-paraffins, iso-paraffins, naphthenes, and aromatics. The product distribution shifted to C5-C18 hydrocarbons at 340° C. Oxygenate concentration reduced by 85% compared with reaction at 260° C. Oxygenates at the highest concentration were hexanone (2.3 wt %) and tetrahydropyran (1 wt %).

TABLE IV
Hydrocarbons present in organic products at 260°
C., 270° C., and 340° C.

| | 260° C. | 270° C. | 340° C. |
|---|---|---|---|
| Compound | (wt %) | (wt %) | (wt %) |
| Hexane | 1.1 | 3.3 | 3.2 |
| Heptane | — | — | 0.4 |
| Octane | — | 0.7 | 1.6 |
| Nonane | 0.6 | 1.0 | 2.4 |
| Decane | 0.1 | 1.5 | 2.6 |
| Undecane | 0.2 | 1.5 | 5.3 |
| Dodecane | 0.4 | 2.8 | 2.2 |
| C13-C18 n-paraffins | 0.1 | 0.4 | 0.8 |
| C6-C12 Iso paraffins, | 20-30 | 30-40 | 70 |
| naphthenes, and aromatics | | | |

Hexane and pentane were also present in the gas phase

The observed low concentrations of aldehydes may be due to the dominant role of decarbonylation and/or rapid hydrogenation of aldehydes to primary alcohols. Low molecular weight alcohols, such as ethanol, propanol, and butanol, observed at lower temperatures (260-270° C.) may be formed via C—C hydrogenolysis of isosorbide followed by dehydration and C—O hydrogenolysis reactions. According to the GC-MS data, primary alcohols are dominant over secondary alcohols. This may be the result of easier dehydration of secondary alcohols over primary alcohols. With increasing temperature, the chemistry becomes more complex due to activation of other chemical transformations such as cracking. Isosorbide thermal decomposition initiates at T>270° C. The increase in the distillate fraction at 340° C. may be a combination of olefin oligomerization, aldol condensation, etherification of alcohols, and aromatization leading to heavy alkyl aromatics. A variety of distillates may be produced by modifying the oxygenate feedstock, temperature, residence time, and other reaction parameters. Dependent upon feedstock, market needs, and equipment parameters, different fuel range distillates may be produced.

### Example 2

### Hydrocondensation Plus Hydrodeoxygenation

As described with respect to FIGS. 1 through 3, it was recognized that the organic products from single stage sorbitol hydrotreating over Pt/Pd catalyst had significant quantities of undesirable oxygenates when the reaction temperature was 270° C. or less. Thus, Example 2 provides data for hydrocondensation in combination with hydrodeoxygenation to eliminate all oxygen. A mixture of oxygenates and hydrocarbons was collected by hydrotreating 40 wt % sorbitol over Pt/Pd catalyst at 270° C. This mixture was hydrodeoxygenated in a second stage over either the same sample of Pt/Pd catalyst or conventional hydrodeoxygenation catalyst at 340° C., 1,200 psig, and 0.6 h⁻¹ (WHSV).

Table V shows density and elemental composition of products. Combined hydrocondensation with hydrodeoxygenation provides improved the product quality. The density of organic products decreased to 0.72 from 0.85 while the carbon content increased to 85 wt % from 73 wt %. Oxygen in the product was reduced from 14 wt % to less than 0.2 wt %. Oxygen originating from oxygenates was 0.06 wt % (detected by GC-AED for oxygen) while that from water was 0.01 wt %. This divergence was probably due to measurement error.

TABLE V
Product quality obtained after hydrocondensation alone
and hydrocondensation with hydrodeoxygenation.

| | | Den- | | | | O from |
|---|---|---|---|---|---|---|
| | Temp | sity | C | H | O | oxygenates |
| Process | ° C. | g/cc | wt % | wt % | wt % | wt % |
| Hydrocondensation | 270 | 0.85 | 73 | 12.7 | 14.3 | 14 |
| Hydrocondensation | 270 & | 0.72 | 84.8 | 14.9 | <0.02 | 0.06 |
| Plus Hydro- | 340 | | | | | |
| deoxygenation | | | | | | |

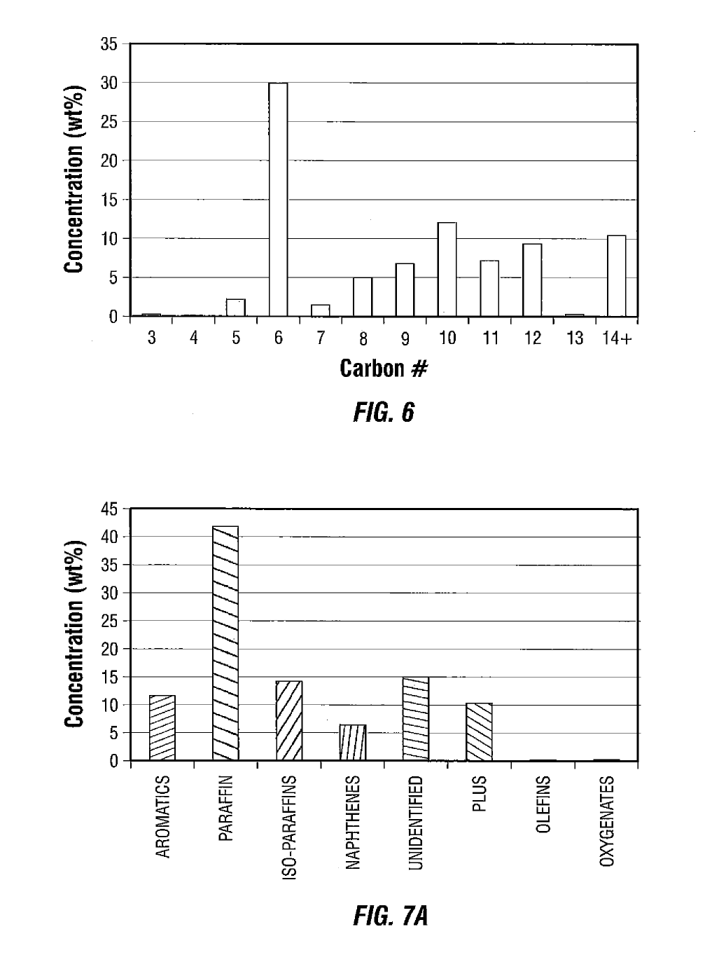

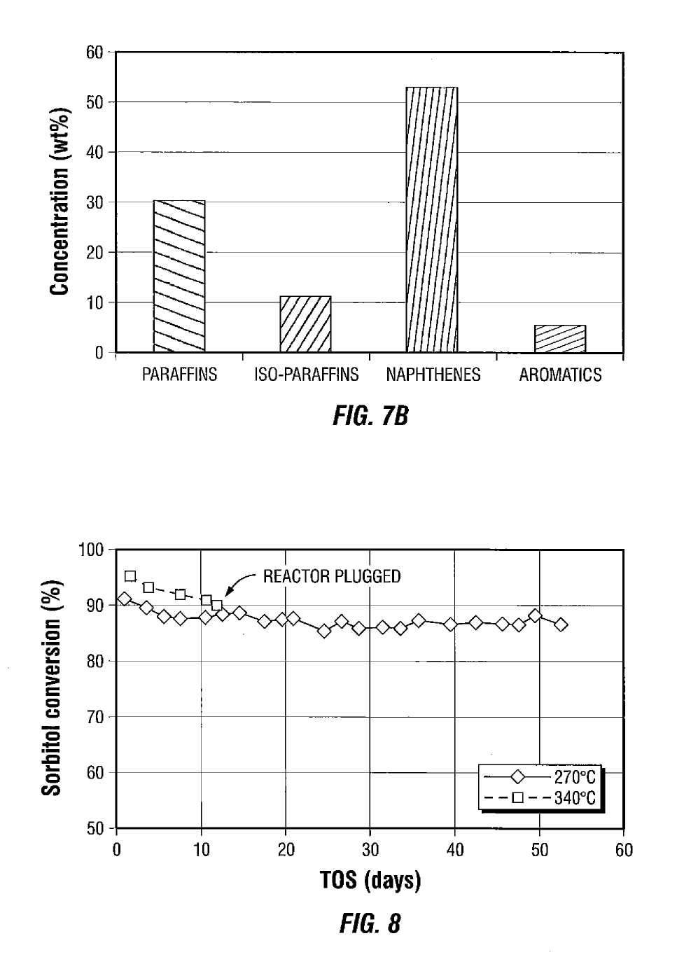

Detailed hydrocarbon analysis (DHA) of the product identified hydrocarbons and estimated fuel properties. A distribution of products as a function of carbon number and type of hydrocarbons is shown in FIGS. 6, 7 and 7A. The product has hydrocarbons in the range of C5-C14, as shown in FIG. 6. Because DHA is a technique for analysis of gasoline boiling compounds, all hydrocarbons with carbon number greater than 14 are represented as C14+ in FIG. 6. FIG. 7 (which is data from early development of the process) indicates that the product was mainly paraffinic with n-hexane being the predominant compound (30 wt %). The remaining 70 wt % of the product had C7-C14 paraffins, iso-paraffins, naphthenes, and aromatics. No oxygenates or olefins were detected by DHA. Furthermore, the final product did not have any sulfur or benzene. GC-MS analysis confirmed these results. Data from later testing shown in FIG. 7A shows high naphthene make with correspondingly reduced paraffins, iso-paraffins and aromatics. As described above, high naphthene make is potentially attractive in jet, diesel and gas oil.

Polishing may be accomplished with a variety of hydrodeoxygenation catalysts. Differences in oxygen removal are negligible with different hydrodeoxygenation catalysts (Table VI). In all cases, polishing with hydrodeoxygenation catalysts reduced oxygen content from ˜14 wt % to less than 1 wt %. Oxygen levels below 0.5 wt %, including less than approximately 0.25 wt % are sufficient for stable fuel range hydrocarbons. In many cases oxygen levels were well below 0.05 wt % with polished hydrocarbons having less than approximately 0.04% or 0.02%. The polished hydrocarbons make an ideal hydrocarbon fuel and were characterized to determine fuel properties.

TABLE VI
Polishing with hydrodeoxygenation catalysts

| | Polishing step | | Oxygen in |
|---|---|---|---|
| | temperature | WHSV | the product |
| Catalyst | ° C. | per h | wt % |
| HPC - 1 | 340 | 0.4 | <0.02 |
| HPC - 2 | 345 | 0.6 | 0.23 |
| HPC - 3 | 350 | 0.4 | <0.4 |

Fuel properties for naphtha are shown in Table VII. The DHA analysis of the gasoline fraction indicated that n-pentane and n-hexane accounted for 20% of that fraction, which resembles natural gasoline. The remaining 80% of the gasoline fraction was composed of paraffins, iso-paraffins, naphthenes, and aromatics. No olefins were present in the product, and the amount of benzene present was below the detection limit. The lack of benzene is important because future gasoline regulations restrict the amount of benzene in gasoline. Other properties of this fraction are shown in Table VII. As seen from this table, density, heat of combustion, and total acid numbers meet or exceed specifications.

TABLE VII
Fuel properties of the Naphtha Fraction
from Example 2 Hydrocondensation.

| | Naphtha | Specification |
|---|---|---|
| Paraffins (wt %) | 35 | |
| Iso paraffins (wt %) | 30 | |
| Naphthenes (wt %) | 14 | |
| Aromatics (wt %) | 9 | |
| Avg. Mol. Wt. | 103 | |
| Density (D4052@60° F., g/cc) | 0.74 | <0.9 |
| SIMDIS D2887 | | |
| T10, ° F. | 144 | |
| T50, ° F. | 350 | 170-250 |
| T90, ° F. | 385 | 250-365 |
| Sulfur (ppm) | <1 | |
| Oxidative Stability by D525, min | >300 | |
| Gross heat of combustion (Btu/lb) | 20304 | ~20000 |
| Net heat of combustion (Btu/lb) | 19026 | |
| TAN by D664 (mg KOH/g) | 0.11 | ~0.1 |
| Copper strip corrosion by D130 | 1a | |
| Gum by D381 (unwashed), mg/100 ml | <4 | |

As demonstrated in Table VII these fuels have a distributed range of paraffins, iso-paraffins, naphthenes, and aromatics. With an overall density of approximately 0.75 and low sulfur content, renewable fuels purified using the techniques described herein are ideal for use as gasoline engine fuels. Unlike ethanol and other alcohol based fuels, these renewable fuels have a high heat of combustion and deliver equivalent energy to that of traditionally purified hydrocarbons.

A renewable jet fuel has been refined and isolated using the procedures, methods and systems described herein. This fuel has favorable properties for a jet fuel and may be used as a fungible substitute for fuels obtained from other hydrocarbon resources. Table VIII further confirms that each fuel property is within the standards set for commercial Jet A and JP-8 standards. Note that freeze point for the renewable fuel is well below the standard required for Jet A and JP-8. Several experiments based on described Example 2 produced several jet samples as shown as Sample A Jet, Sample B Jet and Sample C Jet where hydrodeoxygenation and cutpoints in the distillation column created jet products with slightly different properties. The specifications for Jet A and JP-8 are also shown. In every property measured the Renewable jet fuel meets or exceeds the Jet A standard required for commercial fuels.

TABLE VIII
Jet Fuel Properties

| | Sample | | | | | | |
|---|---|---|---|---|---|---|---|
| | Sample A | Sample B | Sample C | | | | |
| | Jet | Jet | Jet | Jet A | | JP-8 | |
| Oxygen by AED | 0.07 | 0.375 | nd | | | | |
| Gravity by D1298, deg API | 36.95 | 38.17 | 41.06 | 37 | (min) | 37 | (min) |
| | | | | 51 | (max) | 51 | (max) |
| Density @60 F. by D4052, | 0.8393 | 0.833 | 0.8192 | 0.775 | (min) | 0.775 | (min) |
| g/cc | | | | 0.84 | (max) | 0.84 | (max) |
| Total acidity by D3242, | | <0.05 (D664) | <0.05 | 0.1 | (max) | 0.015 | (max) |
| mgKOH/g | | | | | | | |
| Freeze point by D5972, deg C. | −39.2 | −70 | −39.9 | max | −40 | max | −47 |
| Gum, existent by D381, | | | 2 | 7 | (max) | 7 | (max) |
| mg/100 ml | | | | | | | |
| Sulfur by D2622, ppmw | 2 (by | | <1 | 440-2900 | | 0.3 | (max) |
| | XRF) | | | | | | |
| sulfur wt % mercaptan by | | | 0.0001 | 0.003 | (max) | 0.002 | (max) |
| D3227, wt % | | | | | | | |
| Color, Saybolt by D156 | | | | Min | +16 | Report | |
| Corrosion, CST 2 hr @ 212 F. | 1a | | 1b | 1 | (max) | 1 | (max) |
| by D130 | | | | | | | |
| MSEP by D3948 | | | 98 | 85 | (min) | 80 | (min) |
| Hydrogen content by D3701, | 13.44 | | 14.15 | | | 13.4 | (min) |
| wt % | | | | | | | |
| Aromatics by D1319, vol % | | | 0 (By | 25 | (max) | 25 | (max) |
| | | | D5186) | | | | |
| Olefins by D1319, vol % | | | 0 (By | | | 5 | (max) |
| | | | D6550) | | | | |
| Net heat of comb by D3338, | 18,386 | 18,561 | 18671 | 18,400 | (min) | 18,400 | (min) |
| btu/lb | | | | | | | |
| Flash point by D56, deg F. | 149 (D93) | 140 (D93) | 126.5 | 110 | (min) | 100 | (min) |
| Viscosity at −20 C. by D445, | 2.418 | | 5.996 | 8 | (max) | 8 | (max) |
| cSt | | | | | | | |
| Viscosity at 104 F. by D445, | 1.887 | | 1.562 | 1.3 | (min) | | |
| cSt | | | | 1.9 | (max) | | |
| Conductivity by D2624, pSm | | | 2 | Report | | 150 | (min) |
| | | | | | | 600 | (max) |
| Thermal stability by D3241 | | | | | | | |
| (JFTOT) | | | | | | | |
| Pressure drop, mm Hg | | | 0 | 25 | (max) | 25 | (max) |
| Tube deposit code | | | <1 | <3 | | <3 | |
| Distillation by D86, vol % | | | | | | | |
| deg F. | | | | | | | |
| IBP | 366.8 | 344.5 | 336 | | | Report | |
| T10 | 393.6 | 372.9 | 368.8 | 400 | (max) | 401 | (max) |
| T50 | 429.8 | 417.2 | 411.6 | Report | | Report | |
| T90 | 507.9 | 515.5 | 501.8 | 550 | (max) | Report | |
| End point | 537.8 | 548.6 | 548.1 | 572 | (max) | 572 | (max) |
| Residue | 1.2 | 1.3 | 1.2 | 1.5 | (max) | 1.5 | (max) |
| Loss | 0.4 | 0.6 | 0.6 | 1.5 | (max) | 1.5 | (max) |
| Combustion | | | | | | | |
| Smoke point by D1322, mm | | | 31 | 25 | (min) | 25 | (min) |
| OR | | | | | | | |
| Smoke point by D1322, mm | 19 | | 31 | 18 | (min) | 18 | (min) |
| AND | | | | | | | |
| Naphthalene by D1840, vol % | 0.52 (wt %) | | 0 | 3 | (max) | 3 | (max) |
| Carbon residue on 10% | | | <0.10 | 0.15 | (max) | | |
| bottoms by D524 | | | | | | | |
| Ash by D428, wt % | <0.001 | | 0.001 | 0.01 | (max) | | |
| Cetane index by D4737 | 39 | 40 | 43 | 40 | (min) | Report | |
| Particulate by D5452, mg/L | | | 0.0006 | Report | | 1 | (max) |
| Appearance by D4176 | Clear & | Clear & | Clear & | Clear & | | Clear & | |
| | bright | bright | bright | bright | | bright | |
| Karl Fischer water by | 39 | | 70 | | | Report | |
| D6304, ppm | | | | | | | |
| Water reaction by D1094 | | | | | | | |
| Volume change, ml | 0 | | 0 | Report | | | |
| Separation rating | 2 | | 1 | 2 | (max) | | |
| Interface rating | 1b | | 1 | 1b | (max) | 1b | (max) |
| Lubricity by D6079 HFRR, | | | 632.5 | | | | |
| micron | | | | | | | |
| Halides by IC, ppmw | | | | | | | |
| Chloride | | | <0.1 | | | | |
| Simulated distillation by | | | | | | | |
| D2887, wt % deg F. | | | | | | | |
| IBP | 293.6 | | 244 | | | | |
| T10 | 360.7 | | 335.3 | | | | |
| T50 | 426.6 | | 410.7 | | | | |
| T90 | 537.4 | | 530.1 | | | | |
| End point | 580.4 | | 596.4 | | | | |
| NOISE | | | | | | | |
| Paraffins, wt % | 3.3 | | 2.5 | | | | |
| Iso paraffins, wt % | 9 | | 7.3 | | | | |
| One ring naphthenes, wt % | 36 | | 41.6 | | | | |
| Two ring naphthenes, wt % | 32.5 | | 42.5 | | | | |
| Three ring naphthenes, wt % | 6.3 | | 5.8 | | | | |
| Total naphthenes | 74.8 | | 89.9 | | | | |
| Aromatics, wt % | 13 | | 0.3 | | | | |
| C/H ratio | 0.53 | | 0.52 | | | | |
| Avg. molecular weight | 176.4 | | 172.3 | | | | |
| Combustion | | | | | | | |
| C | 85.58 | 85.67 | 85.83 | | | | |
| H | 13.39 | 13.02 | 13.58 | | | | |
| N | 0 | 0 | 0 | | | | |
| S | 0 | 0 | 0 | | | | |

| Metals analysis by | |
|---|---|
| UOP 389 method | Conc. |
| Metal | (ppmw) |
| Al | 0.03 |
| Ca | 0.03 |
| Co | <0.02 |
| Cr | <0.02 |
| Cu | <0.02 |
| Fe | 0.11 |
| K | <0.02 |
| Mg | 0.02 |
| Mn | <0.02 |
| Mo | <0.02 |
| Na | 0.04 |
| Ni | <0.02 |
| P | 0.03 |
| Pb | <0.02 |
| Pd | <0.02 |
| Pt | <0.02 |
| Sb | <0.02 |
| Sr | <0.02 |
| Ti | <0.02 |
| V | <0.02 |
| Zn | 0.02 |

*MSEP: Micro Separometer test to determine water separation characteristic of kerosene fuel

Table IX shows fuel properties of sample diesels produced by Example 2. The NOISE analysis of the diesel fraction indicates that it is mainly composed of paraffins, iso-paraffins, and naphthenes, as shown in Table IX. The amount of naphthenes in hydrocondensation diesel is twice the amount in ULSD, the amount of paraffins is ⅕, and aromatics are 1/10 of the amount in ULSD. This unique product distribution resulted in excellent cold flow properties compared to ULSD. The cloud point and pour point of the distillate fraction were −66 and −70° F. indicating that this biomass based diesel fuel may be used at or below 60° F. The cetane number of the hydrodeoxygenation-diesel measured through a blended IQT test is ˜58, superior to conventional ULSD. Other properties such as density, API gravity, lubricity, heat of combustion, and total acid number (TAN) are similar to that of ULSD. The distillation profile (T10, T50, and T90 points) also resembles that of ULSD. Furthermore, the amount of sulfur is <1 ppm. This superior quality on-spec diesel can be directly blended into the existing ULSD pool as a drop in fuel. The measured flash, pour and cloud point of distillate fraction are superior to that of ULSD.

TABLE IX
Diesel Fuel Properties

| | | Sample B | Sample C | | | | | |
|---|---|---|---|---|---|---|---|---|
| | Sample A | Diesel in | Diesel from | | | | | |
| | Diesel in | Lab using | Pilot Plant | Sample D | Sample E | Sample F | Spec for | |
| | Lab using | expected | using expected | Diesel from | Diesel from | Diesel from | No 2 | |
| | ideal feed | natural feed | natural feed | Pilot Plant | Pilot Plant | Pilot Plant | diesel | |
| Oxygen by | | | 0.2 | 0.73 | 0.6 | 0 | | |
| AED-O, wt % | | | | | | | | |
| Oxygen by | | | | 1.87 | 0.96 | 0.45 | | |
| combustion, | | | | | | | | |
| wt % | | | | | | | | |
| NOISE | | | | | | | | |
| Paraffins, | 7 | 4 | 6 | 3 | 3.6 | 1.7 | | |
| wt % | | | | | | | | |
| Iso paraffins, | 18 | 16 | 15 | 7 | 8.2 | 5.2 | | |
| wt % | | | | | | | | |
| One ring | 40 | 40 | 38 | 29 | 35.4 | 36.4 | | |
| naphthenes, | | | | | | | | |
| wt % | | | | | | | | |
| Two ring | 30 | 31 | 29 | 32 | 30.4 | 44.43 | | |
| naphthenes, | | | | | | | | |
| wt % | | | | | | | | |
| Three ring | 5 | 6 | 7 | 10 | 7.7 | 10.85 | | |
| naphthenes, | | | | | | | | |
| wt % | | | | | | | | |
| Total | 73.99 | 78.18 | 74 | 71 | 73.5 | 91.68 | | |
| naphthenes | | | | | | | | |
| Aromatics, | 0.22 | 2 | 5 | 20 | 14.5 | 1.4 | ~35% | |
| wt % | | | | | | | | |
| C/H ratio | 0.51 | 0.51 | 0.52 | 0.55 | 0.54 | 0.53 | | |
| Avg. | 182 | 199 | 198.8 | 186.7 | 182.6 | 181.7 | | |
| molecular | | | | | | | | |
| weight | | | | | | | | |
| Cetane number | 58 | | 52.14 | | | 45.22 | >40 | |
| by IQT D6890 | | | | | | | | |
| Cloud point | −74 | −66 | −76 | | <−76 | <−76 | | |
| D5773, ° F. | | | | | | | | |
| Pour point | −88 | −70 | | <−60 (by | <−70.6 | <−70.6 | | |
| D5949, ° F. | | | | D97) | | | | |
| CFPP by | | | | | >−60 | −62.5 | | |
| D6371, ° F. | | | | | | | | |
| Flash point by | | | 182 | 166.1 | 144 | 120 | 125 | (min) |
| D93 - closed | | | | | | | | |
| cup, ° F. | | | | | | | | |
| Density D4052 | | 0.8279 | 0.8275 | 0.8629 | 0.851 | 0.83297 | | |
| @60 F., g/cc | | | | | | | | |
| API Gravity | | 39.25 | 39.33 | 32.32 | 34.61 | 38.21 | | |
| D4052@60 F., | | | | | | | | |
| deg API | | | | | | | | |
| Copper strip | | | | 1a | 1a | 1a | 3 | |
| Corrosion by | | | | | | | | |
| D130 | | | | | | | | |
| Distillation by | | | | | | | | |
| D86 (based on | | | | | | | | |
| vol %) | | | | | | | | |
| T10, ° F. | | | 415.8 | 390.6 | 375.4 | 369.3 | | |
| T50, ° F. | | | 452.8 | 446.4 | 437.2 | 431.1 | | |
| T90, ° F. | | | 593.7 | 590.9 | 593.4 | 569.1 | 540 | (min) |
| | | | | | | | 640 | (max) |
| SIMDIS D2887 | | | | | | | | |
| (based on wt %) | | | | | | | | |
| T10, ° F. | | 414 | 398 | 365 | 347.1 | 337.5 | | |
| T50, ° F. | | 489 | 456.7 | 463.5 | 439.2 | 425.1 | | |
| T90, ° F. | | 574 | 623.7 | 621.7 | 618.6 | 591.3 | 572 | (min) |
| | | | | | | | 673 | (max) |
| Lubricity by D | | 361 | 517 | 349 | 456 | 618.5 | 520 | (max) |
| 6079 (HFRR), | | | | | | | | |
| micron | | | | | | | | |
| Gross heat of | | 19946 | | 19333 | 19543 | 19807 | | |
| combustion, | | | | | | | | |
| Btu/lb | | | | | | | | |
| Net heat of | | 18665 | | 18200 | 18385 | 18569 | | |
| combustion, | | | | | | | | |
| Btu/lb | | | | | | | | |
| TAN by D664, | 0.21 | | | | 0 | 0.34 | | |
| mg KOH/g | | | | | | | | |
| Sulfur, ppm | <1 | <1 | | <1 | 1 | <1 | 15 | |
| Oxidative | | | | 0 | 0 | 0 | 0.05 | (max) |
| stability, hours | | | | | | | | |
| Water and | | | | | | | | |
| sediment, vol % | | | | | | | | |
| Kinematic | | | | 2.271 | | 1.857 | 1.9-4.1 | |
| viscosity at 40 | | | | | | | | |
| C., mm2/s | | | | | | | | |
| Na and K, | | | | 5.65 | 1.5 | 3 | | |
| combined, | | | | | | | | |
| ppmw | | | | | | | | |
| Ca and Mg, | | | | 1.4 | 1.4 | 0.5 | | |
| combined, | | | | | | | | |
| ppmw | | | | | | | | |
| Moisture, KF | | | | | 91 | 64 | | |
| titration, ppm | | | | | | | | |
| Ash, wt % | | | | 0.006 | <0.001 | 0.001 | 0.01 | (max) |
| Conradson | | | | | <0.1 | <0.1 | | |
| carbon residue | | | | | | | | |
| by D4530, wt % | | | | | | | | |
| MSEP by | | | | | | 98 | | |
| D3948 | | | | | | | | |
| Aromatics by | | | | | | 2.53 | | |
| SFC D5186, | | | | | | | | |
| area % | | | | | | | | |

| | Conc. | Conc. | Conc. |
|---|---|---|---|
| Element | (ppmw) | (ppmw) | (ppmw) |
| Al | 0.614 | 0.41 | <0.144 |
| Ba | 0.486 | 0.583 | <2.03 |
| Ca | 0.854 | 0.992 | <0.563 |
| Cd | 0.232 | <1.07 | <1.18 |
| Cr | 0.229 | 0.953 | <0.994 |
| Cu | 0.245 | 0.335 | <2.05 |
| Fe | 0.753 | 0.809 | <2.06 |
| K | 4.06 | <1.09 | <2.03 |
| Mg | 0.5 | 0.361 | <0.046 |
| Mn | 0.051 | 0.376 | <1.69 |
| Mo | 0.846 | 0.565 | <1.74 |
| Na | 1.59 | 0.466 | <1.06 |
| Ni | 0.922 | 0.588 | <1.08 |
| P | 2.57 | 3.05 | <0.611 |
| Si | 4.66 | 0.663 | <1.55 |
| Sr | 0.572 | 0.52 | |
| Ti | 0.956 | 0.622 | <1.74 |
| V | 0.69 | 0.28 | <2.02 |
| Zn | 0.185 | 0.61 | <0.024 |

Table X is a summary of gas oil properties showing the density, API gravity, distillation ranges, and total acid for the hydrocondensation purified gas oil. Gas oil produced by this process is well within the properties of standard gas oils.

TABLE X
Generalized properties of Gas Oil Fraction

| | |
|---|---|
| Density at 60 F., g/cc | 0.91-0.95 |
| API Gravity, deg API | 17-23 |
| Oxygen from AED-O, wt % | <0.2 |
| Simulated Distillation D2887, based on wt % of product | |
| T10, ° F. | 578-711 |
| T50, ° F. | 644-786.6 |
| T90, ° F. | 757-918 |
| Total Acid Number by D664, mg KOH/g | <0.07 |
| NOISE analysis | |
| Paraffins, wt % | 0-5 |
| Iso paraffins, wt % | 0-5 |
| One ring naphthenes, wt % | 0-20 |
| Two ring naphthenes, wt % | 0-30 |
| Three ring naphthenes, wt % | 5-30 |
| Mono aromatics, wt % | 8-35 |
| Di aromatics, wt % | 12-40 |
| Tri aromatics, wt % | 5-15 |
| Tetra aromatics, wt % | 0-4 |
| Kinematic Viscosity at 104 F. by ASTM D445, | 16-210 |
| mm2/s (cSt) | |
| Refractive index by D1218 at 67° C. | 1.5132 |
| D 2622 sulfur, wt % | 0.0005 |
| D 4530 CCR, wt % | <0.21 |
| D5762 total nitrogen, ppm | <7 |
| D 661 Aniline point, deg F. | 119-138 |

Table XI below shows the properties for three specific samples of gas oil made by the Example 2 process.

TABLE XI
Specific Gas Oil Properties

| | Sample | Sample | Sample | Comparative | Comparative |
|---|---|---|---|---|---|
| | A Gas | B Gas | C Gas | Vacuum | Vacuum |
| | Oil | Oil | Oil | Gas Oil 1 | Gas Oil 2 |
| Density at 60 F., g/cc | 0.95 | 0.914 | 0.9538 | | |
| API Gravity, deg API | 16.97 | 23.1 | 16.7 | 22.6 | 29.6 |
| Oxygen from AED-O, | 0.06 | 0.62 | 0.19 | | |
| wt % | | | | | |
| Simulated Distillation | | | | | |
| D2887, based on wt % of | | | | | |
| product | | | | | |
| T10, ° F. | 711.2 | 578.4 | 701.8 | 576 | 548 |
| T50, ° F. | 786.6 | 644.7 | 784.7 | 801 | 728 |
| T90, ° F. | 901.7 | 757.8 | 918 | 958 | 925 |
| Total Acid Number by | 0.05 | 0.06 | 0.07 | | |
| D664, mg KOH/g | | | | | |
| NOISE analysis | | | | | |
| Paraffins, wt % | 0 | 0.21 | 0.02 | | |
| Iso paraffins, wt % | 2 | 2.24 | 1.82 | | |
| One ring naphthenes, | 2.8 | 10.8 | 2.1 | | |
| wt % | | | | | |
| Two ring naphthenes, | 5.8 | 19.4 | 4.2 | | |
| wt % | | | | | |
| Three ring naphthenes, | 8.3 | 16.9 | 6.2 | | |
| wt % | | | | | |
| Mono aromatics, wt % | 27.35 | 9 | 35.1 | | |
| Di aromatics, wt % | 35.26 | 13.6 | 37 | | |
| Tri aromatics, wt % | 15.58 | 12.2 | 12.5 | | |
| Tetra aromatics, wt % | 3 | 2 | 1 | | |
| C/H ratio | 0.64 | 0.59 | 0.64 | | |
| Avg. molecular weight | 355.3 | 279.1 | 350.3 | | |
| Kinematic Viscosity at | 209.7 | 15.71 | 187.6 | 53.2 | |
| 104 F. by ASTM D445, | | | | | |
| mm2/s (cSt) | | | | | |
| Refractive index by | 1.5132 | 1.4874 | 1.5132 | | |
| D1218 at 67° C. | | | | | |
| D 2622 sulfur, wt % | 0.0005 | | 0.0005 | 0.202 | 0.0046 |
| D 4530 CCR, wt % | 0.21 | <0.1 | 0.22 | 0.11 | 0 |
| ICP (especially Ni and V) | see | see | see | | |
| | below | below | below | | |
| D5762 total nitrogen, | 2 | 7.7 | 6.4 | 3286 | 0.7 |
| ppm | | | | | |
| D 661 Aniline point, deg | 138 | 135.9 | 119.7 | 164.4 | |
| F. | | | | | |

| | ppmw | ppmw | ppmw |
|---|---|---|---|
| Al | 0.389 | <0.397 | 1.36 |
| Ba | 0.554 | <0.565 | 0.555 |
| Ca | 0.943 | <0.962 | 0.945 |
| Cd | <1.01 | 1.03 | 1.02 |
| Cr | 0.905 | <0.924 | 1.16 |
| Cu | 0.318 | <0.325 | 0.319 |
| Fe | 1.38 | 4.23 | 45.4 |
| K | <1.03 | <1.05 | 1.03 |
| Mg | 0.343 | <0.35 | 0.344 |
| Mn | 0.357 | <0.365 | 0.383 |
| Mo | 0.537 | <0.548 | 0.538 |
| Na | 0.443 | <0.452 | 0.444 |
| Ni | 0.558 | <0.57 | 0.988 |
| P | 3.54 | 3.27 | 2.12 |
| Si | 231 | 73.8 | 151 |
| Sr | 0.494 | <0.504 | 0.496 |
| Ti | 0.591 | <0.603 | 0.593 |
| V | 0.266 | <0.272 | 0.267 |
| Zn | 0.579 | 0.79 | 9.11 |

In summary, a variety of fuel types may be generated and purified from the products of hydrocondensation and polishing. These fuels will allow production of fungible renewable fuel products that are stable, functional, and equivalent to current fuel products used. Analysis of fuels produced demonstrates, unequivocally that these fuels have properties that are equivalent to or superior to fuel products on the market today.

### Example 3

### Lifetime Testing at 270 and 340° C.

In describing the invention, efforts have also been undertaken to add to the robustness of the process including efforts to extend the catalyst life, address issues related to the expected quality of the feedstock as compared to an ideal feedstock that was used in early efforts to develop the technology of the present invention, and optimize the process regarding GHSV through the catalyst bed. The catalyst is exposed to excessive amount of water during the first stage hydrotreating. Because the support of the catalyst is silica-alumina, it is important to determine hydrothermal stability of the support at typical reaction temperatures. To address this question, lifetime of Pt/Pd catalyst for hydrotreating 40 wt % sorbitol solution was studied at 270° C. and 340° C.

The catalyst showed constant conversion during sorbitol hydrotreating at 270° C., as shown in FIG. 8. Sorbitol conversion began at 91% and decreased to 88% during the first six days. After this equilibration period, the activity remained constant at 86% conversion for 50 days. Organic products, both in liquid and vapor phases, retained 90% of the inlet carbon. The organic phase had 14 wt % oxygen in all samples from day 1 to day 52 indicating significant quantities of oxygenates. The deactivation constant was 0.0023 per day that projects a half life >310 days (Table XII). This suggests that a low-cost, fixed-bed process should be feasible for sorbitol hydrotreating over Pt/Pd catalyst.

TABLE XII
Comparison of deactivation constant and projected half
life of hydrocondensation-catalyst at 270 and 340° C.

| Temperature | 270° C. | 340° C. |
|---|---|---|
| Time-on-stream (days) | >50 | 15 |
| Deactivation constant k_{d} (day⁻¹) | 0.002 | 0.08 |
| Projected half life (days) | >310 | <30 |

The catalyst showed poor stability because feed coking problems when operated as a single stage hydrocondensation reactor at 340° C. Severe deactivation occurred, and the reactor plugged in 14 days (FIG. 8). The deactivation constant was 0.08 per day that predicts a half life of <30 days (Table XII). Fixed-bed processes are typically not viable with a 30-day lifetime. Hydrocondensation followed by hydrodeoxygenation at 340° C. will achieve longer lifetimes than hydrocondensation at 340° C. due to the increased thermal stability afforded by hydrocondensation at 270° C.

### Example 4

### Natural Biomass Oxygenate Conversion

A silica-alumina supported platinum-palladium (PtPd/SiO₂—Al₂O₃) hydrocondensation catalyst was used to convert biomass oxygenates to hydrocarbon fuel grade products. A polishing step using a conventional hydrodeoxygenation catalyst further reduced oxygen content below 1%. A raw biomass derived sorbitol-xylitol (60 wt % total) feed was used as an oxygenate feedstock. Beside sugar alcohols, the feed consisted of 0.6 wt % oligosaccharides, 5 ppm of metals, and <1 ppm of sulfur. The detailed analysis of the as received feed is shown in Table XIII. This feed was diluted to 40 wt % sugar alcohols using distilled water.

TABLE XIII
Detailed analysis of raw corn fiber (60%
dissolved solids) sugar alcohol feed
Sugar alcohols

| | Xy- | | Man- | Sor- | | |
|---|---|---|---|---|---|---|
| Erythritol | litol | Arabitol | nitol | bitol | Dulcitol | Maltitol |
| (wt %) | (wt %) | (wt %) | (wt %) | (wt %) | (wt %) | (wt %) |
| 0.39 | 10.7 | 11 | 3.8 | 21 | 6.7 | 0.04 |

Metals

| P | S | Ni | Mg | Ca | Na | Mn |
|---|---|---|---|---|---|---|
| (ppmw) | (ppmw) | (ppmw) | (ppmw) | (ppmw) | (ppmw) | (ppmw) |
| 3.01 | nd | 1.93 | 0.057 | 0.12 | 1.91 | 0.094 |

Residual sugars

| Malt- | Levo- | Su- | | Glu- | Xy- | Iso- |
|---|---|---|---|---|---|---|
| ose | glucosan | crose | Manose | cose | lose | sorbide |
| (ppmw) | (ppmw) | (ppmw) | (ppmw) | (ppmw) | (ppmw) | (ppmw) |
| 143 | 997 | 476 | 0 | 202 | 187 | 11,398 |

In the first stage, Pt/Pd extrudates diluted with alundum (1:2 weight ratio) were packed in a ¾″ OD reactor. The catalyst was reduced in the presence of hydrogen by following a standard reduction procedure as previously described. After reduction, the temperature was decreased to 50° C. and the catalyst was wetted with the feed (introduced using an ISCO syringe pump) for 2 hours at a liquid hourly space velocity of 3 h⁻¹. Following this step, the reactor temperature was increased so that temperatures in the top, middle, and bottom sections of the catalyst bed were 270° C., 290° C. and 310° C., respectively. The pressure was increased to 1200 psig. No diesel was co-fed with sorbitol in these experiments while the flow of hydrogen was 250 sccm (hydrogen to sugar alcohol molar ratio of 30). The weight hourly space velocity (WHSV) used in all experiments was 0.4 g feed/g cat/h. For each mass balance, products were collected for at least 24 h to ensure steady conversions. The off-gases from the reactor were analyzed using an Agilent 6890 GC equipped with two detectors (TCD and FID) and two columns (a Carboxen column for permanent gases and HP-1 column for hydrocarbons).

The liquid products collected were split into organic and aqueous phases by gravity separation. The aqueous phase was analyzed for unreacted sorbitol by HPLC. Intermediate oxygenates in that phase were analyzed by the total organic carbon (TOC) method and by GC. The organic phase was analyzed using combustion for elemental analysis. This organic phase was further hydrotreated in a second stage using a hydrodeoxygenation catalyst.

A conventional hydrodeoxygenation catalyst was used for second stage hydrotreating or polishing to reduce cracking selectivity and hydrotreat oxygenates. The catalyst was reduced in the presence of hydrogen prior to polishing of hydrocondensation products at 0.42 h⁻¹ WHSV, 330° C. and 1200 psig. The resulting product was fractionated into naphtha, distillate, and gas oil fractions using a spinning band distillation column. Separation of distillate fraction from the gas oil fraction was conducted under vacuum (2 mmHg). The naphtha and distillate fractions were characterized using combustion for elemental analysis, GC—Atomic Emission Detector (AED) for oxygen, Simulated Distillation by ASTM D 2887 (SIMDIS), and Differential Scanning calorimetry for net and gross heats of combustion. Detailed Hydrocarbon Analysis (DHA) was used to determine molecular types of naphtha fraction while NOISE was used for distillate fraction. The distillate fraction was further analyzed to determine its cloud point (by D5773), pour point (by D5949), density (by D4052), lubricity (by D6079), and cetane number (by IQT D6890). As the amount of distillate fraction available was not enough for a stand-alone cetane test, this product was blended with commercial ULSD (30/70 v/v) to generate a blended cetane number. The actual cetane number of hydrocondensation distillate fraction was then calculated by excluding contribution from ULSD.

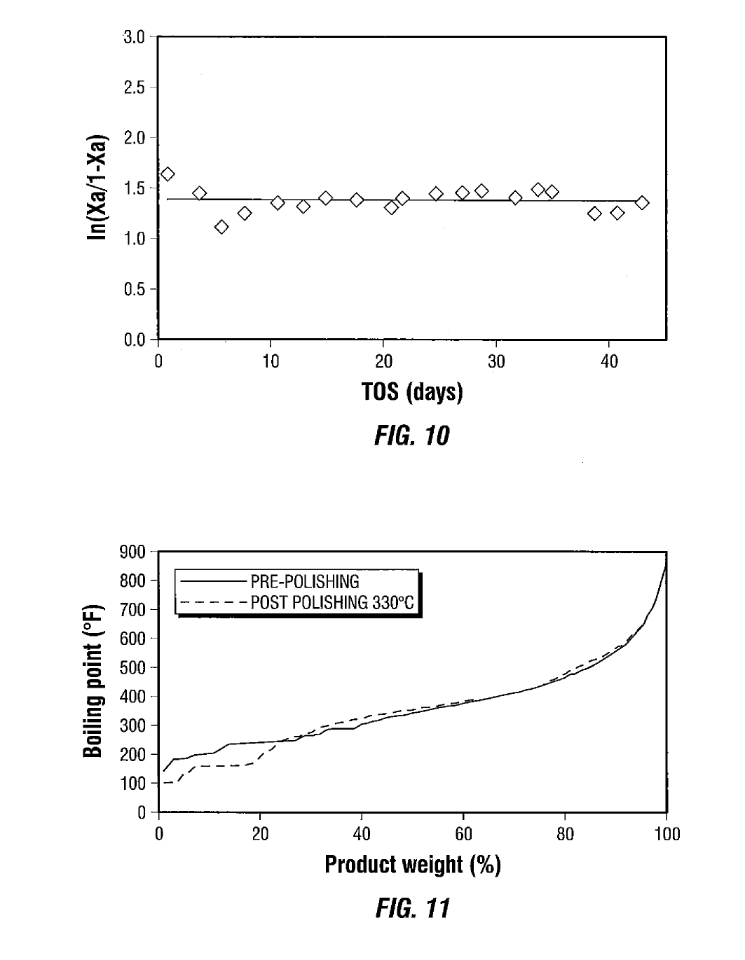

The experiment successfully ran for 43 days (>1000 h) without any plugging or pressure drop problems. The average mass balance throughout the experiment was 96%. The sugar alcohol (C5+C6 alcohols) conversion was >99% throughout the run as shown in FIG. 13. As sugar alcohol conversion was >99%, this data was not useful in determining the deactivation rate. Hence, carbon yield to hydrocarbons and oxygenates (obtained from Total Organic Carbon method) was used to determine catalyst deactivation rate. As shown in FIG. 10, a small initial deactivation was observed during the first 7 days of TOS. However, the catalyst regained its activity and no changes in the activity were observed for the next 35 days. This indicated that the rate of deactivation after initial stabilization was <10%. The amount of oxygen in the organic product was 16% indicating a polishing step was necessary to decrease the oxygen content below 1%. Nevertheless, the catalyst was able to decrease the oxygen content from 52% in the feed to 16% in the product. Previous characterization data indicates that this intermediate product consists of mainly alcohols, ketones, and cyclic ethers with other molecules being C5-C20 hydrocarbons. A simulated distillation profile of this intermediate product is shown in FIG. 11.

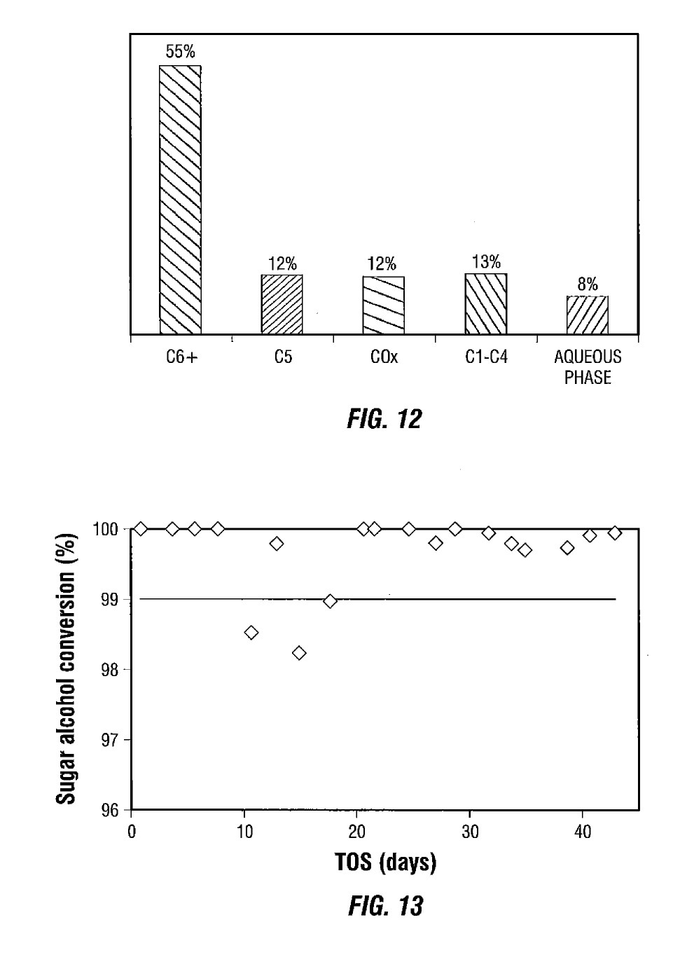

After hydrodeoxygenation of the organic product (obtained from the first step) over conventional hydrodeoxygenation catalyst, the oxygen content of the product decreased from 16% to 0.03%. The product did not contain any sulfur. The overall carbon distribution (including first and second stages) is shown in FIG. 12. About 60% of the carbon was present as C6+ hydrocarbons while 13% of carbon was present as pentane. The light gases from both stages included C1-C4 hydrocarbons and carbon oxides. About 13% of inlet carbon was converted to carbon oxides, which most likely is an overestimated number due to analytical limitations. The overall hydrogen consumption (for both stages) was about 1400-1500 scf/bbl, about 30-40% lower than a hydrodeoxygenation-based process.

The true boiling point curve of the finished product obtained from SIMDIS D2887 is shown in FIG. 11 along with the curve for intermediate product. After comparing the two boiling point curves, it appears that the nature of the curve for heavier molecules did not change indicating oxygen was present predominantly as C5-C6 oxygenates. The hydrocondensation process showed about 5% reduction in volume compared to hydrodeoxygenation-based process, mainly due to the production of higher density naphtha and distillates. The C5-C6 volumetric yield was 50% while diesel yield was 28%. These volumetric yields improve product value compared to a process that produces mostly light naphtha.

As seen earlier, the hydrocondensation-based process generates a product that has higher value compared to the hexane-pentane product mixture obtained from direct hydrodeoxygenation of feedstock in the presence of diesel process. Besides this, the hydrocondensation process also offers some cost saving opportunities. The process does not require a diesel co-feed and all capital and operating costs related to this can be eliminated. The hydrogen exiting the reactor mainly consists of carbon oxides and C1-C4 hydrocarbons. This hydrogen, after a small purge, can be recycled back to either sugar hydrogenation step or hydrotreating step. Also, the overall hydrogen consumption of this process is about 30-40% lower than the hydrodeoxygenation-based process. Because of these two reasons, the amount of fresh hydrogen required will be lower, which will decrease the cost of steam-methane reformer and may also improve the life cycle analysis of the process.

### Example 5

### Effect of Space Velocity on Product Selectivity

Experiments were conducted feeding 40 wt % sorbitol in water over a bed of catalyst containing PtPd on a silica/alumina support at 270° C. and different flow rates to generate products at sorbitol feed weight hourly space velocities (WHSV) between 0.4 and 3.5 h⁻¹. Hydrogen was also fed at a constant gas hourly space velocity (GHSV) of 416 h⁻¹. Gas phase products were quantified using gas chromatography (GC). The organic and aqueous products generated were collected and fed to a bed of a commercial hydrotreating catalyst (second stage) operating in a non-sulfided form between 0.4 h⁻¹ and 0.8 h⁻¹ liquid feed WHSV along with hydrogen at 330° C. The second stage reduced product oxygen content to less than 1 wt %. Additional experiments have operated the second stage hydrotreating unit at space velocities up to 3.0 h⁻¹ using both sulfided and non-sulfided commercial hydrotreating catalysts to achieve the same deoxygenation performance.

Yield (wt %) was calculated as the mass of product formed divided by the sum of the mass of sorbitol fed. Heavy naphtha products were defined as material boiling between 180 and 380° F., distillate products were defined as material boiling between 380 and 650° F., and gas oil products were defined as material boiling between 650 and 1000° F.

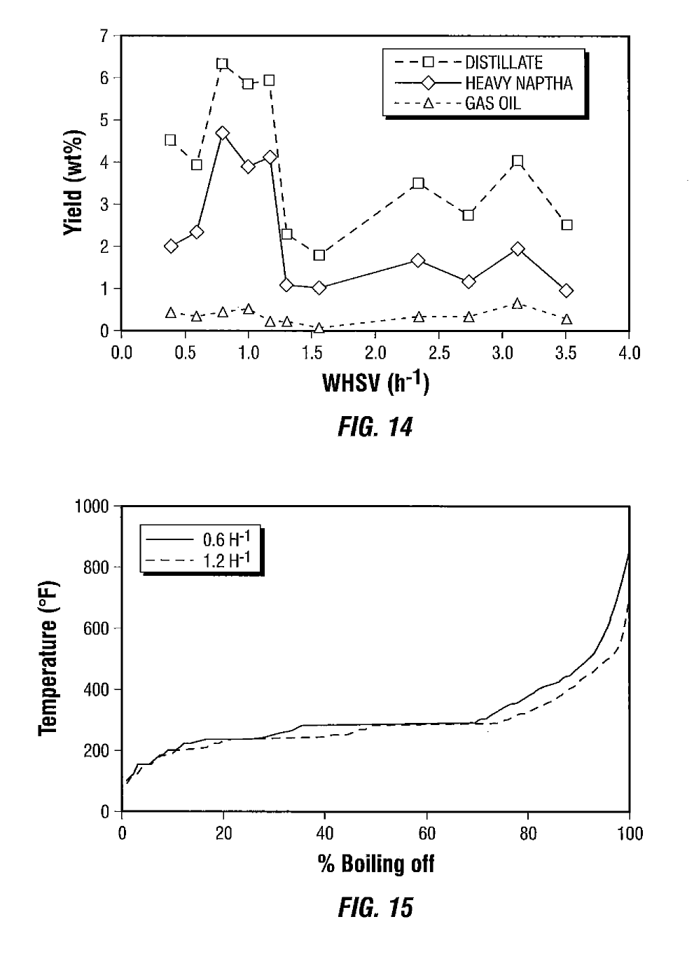

A plot of distillate product yield after deoxygenation by the second stage catalyst versus WHSV contains two maxima (FIG. 14). The first maximum occurs near 1 h⁻¹ and produces between 5 and 6 wt % diesel and the second occurs above 1.6 h⁻¹. Diesel formed at 3.1 h⁻¹ is derived from both organic and aqueous phase intermediates that deoxygenate over the second stage hydrotreating catalyst, whereas the diesel formed at 1 h⁻¹ is primarily derived from the organic intermediate. Similar trends and maxima are observed for the production of naphtha and gas oil. See FIG. 14 and Table XIV for a listing of yields at different conditions tested. All products produced had similar compositional properties regardless of the space velocities used.

TABLE XIV
Yields of Naphtha, Diesel, and Gas oil at Different Conditions

| | | Yields | | |
|---|---|---|---|---|
| 1st stage | 2nd stage | Heavy | | Gas |
| space velocity | space velocity | Naphtha | Distillate | oil |
| 0.39 | 0.44 | 2.02 | 4.52 | 0.45 |
| 0.59 | 0.45 | 2.34 | 3.95 | 0.36 |
| 0.80 | 0.80 | 4.67 | 6.32 | 0.48 |
| 1.00 | 0.57 | 3.90 | 5.87 | 0.54 |
| 1.17 | 0.49 | 4.10 | 5.93 | 0.26 |
| 1.30 | 0.42 | 1.11 | 2.31 | 0.25 |
| 1.56 | 0.42 | 1.04 | 1.79 | 0.10 |
| 2.34 | 0.42 | 1.67 | 3.50 | 0.35 |
| 2.73 | 0.41 | 1.16 | 2.76 | 0.35 |
| 3.12 | 0.41 | 1.96 | 4.03 | 0.67 |
| 3.51 | 0.41 | 0.96 | 2.51 | 0.32 |

### Example 6

### Production of Distillates Using Base Metal Catalyst

Experiments were conducted by feeding 40 wt % sorbitol in water to a conventional base metal hydroprocessing catalyst at 290° C. and at two different sorbitol weight hourly space velocities (WHSV). Hydrogen was also fed at a constant gas hourly space velocity (GHSV) of 750 h⁻¹. Gas phase products were quantified using gas chromatography (GC). The organic products collected were analyzed using Simulated Distillation to quantify the amount of distillates (boiling between 380 to 680° F.) formed in the process.

As shown in FIG. 15, about 20 wt % of the organic product was boiling in the distillate range at 0.6 and 1.2 h⁻¹ space velocities. Naphtha and fuel range molecules were also produced using base metal catalysts.

### Example 7

### Temperature Graded Reactor

A temperature graded bed approach was used to decrease the oxygen content of feed in a single pass and produce fungible hydrocarbon fuels. Sorbitol was used as the model compound to represent cellulosic alcohols. As shown in FIG. 9C, the beginning of the fixed bed catalytic reactor (a) may be at a lower temperature (260-270° C.), the middle of the reactor (c) at intermediate temperature (290-300° C.), and the end of the reactor (e) may be at a higher temperature (320-340° C.). This enables conversion of intermediates formed in the top of the reactor to final hydrocarbon products. The catalyst used in the present invention is a commercial PtPd/SiO₂—Al₂O₃ catalyst. Before the reaction, the catalyst was reduced at 450° C. for 15 h. The reaction was carried out at 1200 psig. The inlet feed consisted of 40 wt % sorbitol in water and hydrogen gas.

Temperature may be graded across one or more reactors that contain one or more catalysts. In one embodiment a single reactor contains a graded temperature from 260 to 340° C. with the temperature increasing across the catalyst. In another embodiment one reactor is maintained between 260-270° C., a subsequent reactor is maintained between 290-300° C. and a final reactor is maintained between 320-340° C. Reactors may be hydrocondensation or hydrodeoxygenation reactors. In one embodiment a single graded reactor contains multiple catalysts and temperature zones. As shown in FIG. 9C, the reactor may contain a guard material (a) to protect the hydrocondensation catalyst (b), a separation material (c) followed by a hydrodeoxygenation catalyst and a retaining material (e). Heating maintains the hydrocondensation catalyst (b) between 250-300° C. and the hydrodeoxygenation catalyst between 320-340° C. Alternatively, separate reactors may be run in series with the first hydrocondensation reactor maintained between 260-270° C., a second optional hydrocondensation reactor maintained between 290-300° C., and a third hydrodeoxygenation reactor maintained between 320-340° C. It may be possible to provide to maintain one or more reactors under a variety of temperature regimes, dependent upon the quantity, volume and source of the biomass oxygenates.

In one example, sorbitol conversion was above 92% during a 33 day TOS experiment. The initial conversion dropped from 98% to 93% at the end of 33 days indicating a small deactivation. The elemental oxygen in the organic products was <1 wt % during the first 7 days of TOS while the concentration stabilized around 5 wt % at steady-state. This demonstrates a dramatic reduction in the amount of oxygen present, oxygen content was reduced from 52 wt % to <5 wt % in products in a single pass. The organic product with the least amount of oxygen (˜5000 ppmw) was analyzed to determine its nature. The detailed hydrocarbon analysis indicated that a majority of the product was in distillate range (C9+). The simulated distillation analysis (SIMDIS by D 2887) supported results of detailed hydrocarbon analysis. According to SIMDIS data, 36 wt % products were boiling in gasoline-range while 61% were in distillate-range. This demonstrates an ability to convert raw biomass oxygenates into fungible naphtha and distillate range fuel products that may be incorporated directly into existing fuel streams or used for blending with lower quality fuels to make higher quality blends.

As a comparison of hydrocondensation with efforts to simply remove oxygen from oxygenates derived from biomass and does not attempt to create carbon-carbon bonds to create longer chain hydrocarbons Table XV shows the overall product yields from a hydrodeoxygenation alone process and hydrocondensation alone process. For the hydrocondensation process, on a carbon basis, 6% of inlet carbon was converted to light gases, 46% to light naphtha, 21% to heavy naphtha, and 10% to distillates. In comparison, the hydrodeoxygenation alone process made more light gases and did not make heavy naphtha and distillates.

TABLE XV
Comparison of Hydrodeoxygenation alone with Hydrocondensation

| | Process | |
|---|---|---|
| Carbon yields (%) | Hydrodeoxygenation | Hydrocondensation A |
| Light gases (C1-C4) | 25 | 6 |
| Light naphtha (C5-C6) | 74 | 46 |
| Heavy naphtha (C7-C10) | 0 | 21 |
| Distillate (C11-C18) | 0 | 10 |
| Carbon Dioxide | 1 | 3 |
| Oxygenates | 0 | 14* |

*Oxygenates may be recycled to the feedstock.

These results indicate that the hydrocondensation using Pt/Pd or conventional hydrodeoxygenation catalyst increases product value over a hexane-pentane product mixture obtained using hydrodeoxygenation alone in the presence of diesel. The process does not require diesel co-feed, which may reduce operating cost. Furthermore, sulfur is completely eliminated from this process. This enables recycling of hydrogen with little purification. All these benefits indicate the increased value of this process.

In closing, it should be noted that the discussion of any reference is not an admission that it is prior art to the present invention, especially any reference that may have a publication date after the priority date of this application. At the same time, each and every claim below is hereby incorporated into this detailed description or specification as an additional embodiment of the present invention.

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims while the description, abstract and drawings are not to be used to limit the scope of the invention. The invention is specifically intended to be as broad as the claims below and their equivalents.

### REFERENCES

All of the references cited herein are expressly incorporated by reference. The discussion of any reference is not an admission that it is prior art to the present invention, especially any reference that may have a publication date after the priority date of this application. Incorporated references are listed again here for convenience:
1. U.S. Pat. No. 5,856,260, U.S. Pat. No. 6,331,575, WO9847617, Mauldin, “Preparation of high activity catalysts” Exxon Res. Eng. Co. (1999).
2. U.S. Pat. No. 6,872,300, US2005075244, US2007191222, Galperin, et al., “Reforming catalyst with chelated promoter.” UOP LLC, (2005).
3. U.S. Pat. No. 6,013,173, U.S. Pat. No. 6,419,820, U.S. Pat. No. 6,495,487, U.S. Pat. No. 6,809,061, U.S. Pat. No. 6,884,340, US2002155946, Bogdan, et al., “Reforming process using a selective bifunctional multimetallic catalyst.” UOP LLC, (2000).
4. U.S. Pat. No. 7,572,925, US2008033188, US20090124839, WO2007146636, WO2008151178, Dumesic and Roman-Leshkov, “Production of Liquid Alkanes in the Jet Fuel Range (C8-C15) from Biomass-Derived Carbohydrates”, Wisconsin Alumni Research Foundation, 2009.
5. U.S. Pat. No. 7,678,950, US20070142633, WO2007075370, Yao, et al., “Process for converting carbohydrates to hydrocarbons.” ConocoPhillips Co. (2007).
6. US2005112739, WO2005040392, Golubkov, “Method for producing hydrocarbons and oxygen-containing compounds from biomass.” Swedish Biofuels AB (2005).
7. U.S. Pat. No. 7,671,246, US2008058563, WO2007103858, Dumesic, et al., “Stable, Aqueous-Phase, Basic Catalysts and Reactions Catalyzed Thereby.” Wisconsin Alumni Research Foundation. (2008).
8. US2009255171, WO2009129019, Dumesic, et al., “Single-Reactor Process for Producing Liquid-Phase Organic Compounds from Biomass.” Wisconsin Alumni Research Foundation, (2009).
9. US20100307050, Sen and Yang, “One-step catalytic conversion of biomass-derived carbohydrates to liquid fuels.” Penn. State Res. Found. (2010)
10. US2011046423, WO2011025632, Sughrue, et al., “Hydrotreating Carbohydrates.” ConocoPhillips Co. (2011).
11. US20110144396, WO2011075322, Lotero, et al., “Process for Converting Biomass to Hydrocarbons and Oxygenates.” ConocoPhillips Co. (2011).
12. US20110087060, WO2011046690, Yao, et al., “Sugar Alcohol Split Injection Conversion.” ConocoPhillips Co. (2011).
13. US20110152513, WO2011078909, Yao, et al., “Conversion of Carbohydrates to Hydrocarbons.” ConocoPhillips Co. (2011).
14. US20110184215, WO2011094325, Jess, et al., “Biomass Pyrolysis In Refinery Feedstock.” ConocoPhillips Co. (2011).
15. U.S. Ser. No. 61/444,004, Anand, et al., filed Feb. 17, 2011, entitled “MoS₂ Catalyst for The Conversion of Sugar Alcohol to Hydrocarbons”
16. WO20100033789, Huber, et al., “Production Of Hydrogen, Liquid Fuels, And Chemicals From Catalytic Processing Of Bio-Oils.” Univ. Massachusetts (2010).
17. EP2179980, Vangelis, “Process for the manufacture of saturated mono- or polycyclic compounds.” Nat & Kapodistrian Univ. (2010).
18. Barron, et al., “The Mechanisms of Hydrogenolysis and Isomerization of Hydrocarbons on Metals: II. Mechanisms of Isomerization of Hexanes on Platinum Catalysts.” J. Catalysis 5:428-45 (1966).
19. Birta, et al., “Kinetic of Sorbitol Decomposition Under Non-isothermal Conditions.” J. Thermal Analysis and calorimetry, 92:635-8 (2008).
20. Bond, et al., “Integrated Catalytic Conversion of g-Valerolactone to Liquid Alkenes for Transportation Fuels” Science 327 (2010) 1110-1114.
21. Chheda, et al., “Liquid-Phase Catalytic Processing of Biomass-Derived Oxygenated Hydrocarbons to Fuels and Chemicals” Angew. Chem. Int. Ed 46:7164-83 (2007).
22. Crossley, et al., “Solid Nanoparticles that Catalyze Biofuels Upgrade Reactions at the Water/Oil Interface.” Science 327:68-72 (2010).
23. Dumesic, Biography, Editorial Board, Journal of Catalysis.
24. Elliott, et al., “Catalytic Upgrading of C5 Feedstocks to Ethylene and Propylene Glycols.” Pacific Northwest National Laboratory,
25. Gürbüz, et al., “Dual-bed Catalyst System for C—C Coupling of Biomass-Derived Oxygenated Hydrocarbons to Fuel-Grade Compounds” Green Chemistry 12:223-7 (2010).
26. Gerritsen, et al., “Ketjenfine 200-A New Aromatics Saturation Catalyst,” Akzo Nobel Courier 36, 1999.
27. Li and Huber, “Aqueous-Phase Hydrodeoxygenation of Sorbitol with Pt/SiO2-Al2O3: Identification of Reaction Intermediates.” J. Catalysis, 270:48-59 (2009).
28. Maris and Davis, “Hydrogenolysis of Glycerol over Carbon-supported Ru and Pt Catalysts.” J. Catalysis 249:328-37 (2007).
29. Mavrikakis, et al., “DFT Studies for Cleavage of C—C and C—O Bonds in Surface Species Derived from Ethanol on Pt(111).” J. Catalysis, 218:178 (2003).
30. Rooney, “The Exchange with Deuterium of Two Cycloalkanes on Palladium Films: π-Bonded Intermediates in Heterogeneous Catalysis.” J Catalysis, 2:53-57 (1963).
31. Snåre, et al., “Heterogeneous Catalytic Deoxygenation of Stearic Acid for Production of Biodiesel.” Ind. Eng. Chem. Res., 45:5708-15 (2006).
32. West, et al., “Catalytic Conversion of Biomass-derived Carbohydrates to Fuels and Chemicals by Formation and Upgrading of Mono-functional Hydrocarbon Intermediates.” Catalysis Today, 147:115-125 (2009).
33. Worldwide Refinery Processing Review, “Monitoring Technology Development and Competition in One Single Source, Second Quarter 2010, Hydrotreating,” Publ. Hydrocarbon Publishing Company, Pennsylvania, (2010).

1. A process for producing longer carbon chain hydrocarbon products from shorter carbon chain oxygenate material, wherein the process comprises:
contacting hydrogen and the oxygenate material with a hydrocondensation catalyst which catalyzes the formation of carbon-carbon bonds in the presence of hydrogen to form longer chain hydrocarbon products.

2. The process according to claim 1 wherein the oxygenate material is derived at least in part from biomass.

3. The process according to claim 1 wherein the oxygenate material is provided in an aqueous solution and the only feedstocks to the process are aqueous and gas.

4. The process according to claim 1 wherein the oxygenate material has an oxygen to carbon ratio of at least 0.6 to one.

5. The process according to claim 1 wherein the oxygenate material includes at least one of: sugar alcohols, sugars, sugar derivatives, hydrogenated sugars, hydrogenated sugar derivatives, glycerol, tetrahydrofurfuryl alcohol, isosorbide, sorbitans, and C3 to C6 polyols and any combination thereof.

6. The process according to claim 1 wherein the oxygenate material contacts the hydrocondensation catalyst at elevated temperature and elevated pressure and the hydrocarbon products include normal paraffins, iso-paraffins and naphthenes.

7. The process according to claim 1 wherein the longer chain hydrocarbon products include some oxygenates and wherein the process further includes the step of contacting hydrogen and the longer chain hydrocarbon products with a hydrodeoxygenation catalyst which catalyzes the removal of oxygen from the longer chain hydrocarbon products.

8. The process according to claim 7 wherein the step of contacting the oxygenate materials with the hydrocondensation catalyst occurs in a first reactor vessel and the step of contacting the longer chain hydrocarbon products with the hydrodeoxygenation catalyst occurs in a second reactor vessel.

9. The process according to claim 7 wherein the step of contacting the oxygenate materials with the hydrocondensation catalyst occurs in a reactor vessel and the step of contacting the longer chain hydrocarbon products with the hydrodeoxygenation catalyst occurs in the same reactor vessel.

10. The process according to claim 7 further including the steps of phase separating the organic phase from the aqueous phase and fractionating the organic phase containing most of the deoxygenated longer chain hydrocarbon products into various distillation fractions.

11. The process according to claim 1 wherein the step of contacting the oxygenate material to the hydrocondensation catalyst produces longer chain hydrocarbon products comprising at least ten weight percent naphthenes.

12. The process according to claim 1 wherein the step of contacting the oxygenate material to the hydrocondensation catalyst produces longer chain hydrocarbon products comprising at least 15 percent naphthenes.

13. The process to claim 1 wherein the catalyst comprises metallic and/or acidic catalyst sites.

14. The process according to claim 1 wherein the catalyst comprises a supported noble metal.

15. The process according to claim 1 wherein the catalyst comprises a supported base metal selected from the group including Ni, Mo, W, Co and combinations thereof.

16. The process according to claim 1 wherein the catalyst further comprises Pt and/or Pd on a support comprising silica and/or alumina.

17. The process according to claim 1 wherein the step of contacting the oxygenate material with a hydrocondensation catalyst comprises contacting the oxygenate material with the hydrocondensation catalyst at a temperature of between 250 to 400° C. at a pressure of between 1000 and 2000 psig where the oxygenate material progresses at a weight hourly space velocity of between 0.4 and 5.0 h⁻¹.

18. A system to convert biomass oxygenates to renewable C5-C25 hydrocarbons comprising:
a. an oxygenate feedstock derived at least in part from biomass and comprising one or more polyols, sugars, or carbohydrates in an aqueous solution;
b. a hydrogen feed;
c. a hydrocondensation reactor including a hydrocondensation catalyst for converting the oxygenate feedstock into longer chain hydrocarbons; and
d. a distillation column for separating the renewable hydrocarbons into product streams comprising C5-C25 range hydrocarbons comprising renewable naphtha, renewable gas oil, and at least one distillate range renewable fuel.

19. The system according to claim 18 further including a phase separator for separating an organic phase including the hydrocarbons from an aqueous phase.

20. The systems according to claim 18 further including a hydrodeoxygenation reactor including hydrodeoxygenation catalyst for removing residual oxygen from the hydrocarbons from in the hydrocondensation reactor and arranged upstream of the distillation column.
